(19) United States
(12) Patent Application Publication
(10) Pub. No.: US 2006/0156490 A1
(43) Pub. Date: Jul. 20, 2006

(54) Symmetrical diazo compounds comprising 2-imidazolium groups and a cationic linker, compositions comprising them, method of coloring, and device

(76) Inventors: Herve David, Ia Varenne Saint Hilaire (FR); Andrew Greaves, Montevrain (FR); Nicolas Daubresse, Ia Celles St Cloud (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/300,512

(22) Filed: Dec. 15, 2005

Related U.S. Application Data
(60) Provisional application No. 60/681,460, filed on May 17, 2005.

(30) Foreign Application Priority Data
Dec. 15, 2004 (FR) 04 53005

(51) Int. Cl. A61K 8/49; C07D 403/02

(52) U.S. Cl. 8/406; 534/769

(57) ABSTRACT

The present invention relates to symmetrical cationic diazo compounds of formula (I) below, their resonance forms, and also their acid addition salts and their solvates: 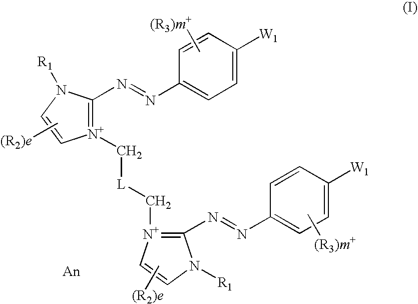
in which W₁ radicals, which are identical, represent a halogen atom or an —NR₄—Ph—NR₅R₆, —NR₄—Ph—OR₇, —O—Ph—OR₇ or —O—Ph—NR₅R₆ group;
L, a cationic linker connecting the two identical azo chromophores represents a C₂-C₄₀ alkyl radical which carries at least one cationic charge and is optionally substituted and/or optionally interrupted by one or more, saturated or unsaturated (hetero)cycles; the linker L containing no azo, nitro, nitroso or peroxo bond. The invention further relates to dyeing compositions comprising such compounds as a direct dye in a medium appropriate for the dyeing of keratin fibres, and also to a method of colouring keratin fibres that employs this composition, and a device having a plurality of compartments.

This application claims benefit of U.S. Provisional Application No. 60/681,460, filed May 17, 2005, the contents of which are incorporated herein by reference. This application also claims benefit of priority under 35 U.S.C. § 119 to French Patent Application No. 04 53005, filed Dec. 15, 2004, the contents of which are also incorporated herein by reference.

The present invention relates to symmetrical cationic diazo compounds containing 2-imidazolium groups and a cationic linker, to dyeing compositions comprising such compounds as a direct dye in a medium appropriate for the dyeing of keratin fibres, to a method of colouring keratin fibres that employs this composition, and to a device having a plurality of compartments.

It is known practice to dye keratin fibres, and especially human keratin fibres such as the hair, with dyeing compositions containing direct dyes. These compounds are coloured, and colouring, molecules having an affinity for the fibres. It is known practice, for example, to use direct dyes of nitrobenzene type, anthraquinone dyes, nitropyridines and dyes of azo, xanthene, acridine, azine or triarylmethane type.

Commonly these dyes are applied to the fibres, optionally in the presence of an oxidizing agent if a simultaneous fibre lightening effect is desired. When the leave-in time has elapsed, the fibres are rinsed, optionally washed, and dried.

The colorations which result from the use of direct dyes are temporary or semi-permanent colorations, because the nature of the interactions which bind the direct dyes to the keratin fibre, and their desorption from the surface and/or the core of the fibre, are responsible for their relatively low tinctorial strength and relatively poor wash resistance or perspiration resistance.

It is known from patent application EP 1377263 to employ particular direct cationic diazo dyes containing two cationic heterocyclic groups. These compounds, although representing an advance in the art, give dyeing results which nevertheless remain capable of improvement.

For the purposes of the present invention, and in the absence of any indication otherwise:
an alkyl(ene) radical or the alkyl(ene) moiety of a radical is linear or branched.
An alkyl(ene) radical or the alkyl(ene) moiety of a radical is said to be substituted when it comprises at least one substituent selected from the following groups:
   hydroxyl,
   C₁-C₄ alkoxy, C₂-C₄ (poly)hydroxyalkoxy,
   amino, amino substituted by one or two identical or different C₁-C₄ alkyl groups which optionally carry at least one hydroxyl or C₁-C₂ alkoxy group, it being possible for said alkyl radicals to form, with the nitrogen atom to which they are attached, a heterocycle containing 5 or 7 ring members which is saturated or unsaturated, is optionally aromatic, is optionally substituted and contains optionally at least one other heteroatom different or not from nitrogen,
   an alkylcarbonylamino radical (R′CO—NR—) in which the radical R is a hydrogen atom or a C₁-C₄ alkyl radical,
   an alkylsulphonyl radical (R—SO₂—) in which the radical R represents a C₁-C₄ alkyl radical,
   an alkylsulphinyl radical (R—SO—) in which the radical R represents a C₁-C₄ alkyl radical,
   an alkylcarbonyl radical (R—CO—) in which the radical R represents a C₁-C₄ alkyl radical.
An aromatic or non-aromatic, saturated or unsaturated (hetero)cyclic radical, or the aromatic or non-aromatic, saturated or unsaturated (hetero)cyclic moiety of a radical, is said to be substituted when it comprises at least one substituent, preferably carried by a carbon atom, selected from:
   an optionally substituted C₁-C₁₆, preferably C₁-C₈, alkyl radical;
   a halogen atom such as chlorine, fluorine or bromine;
   a hydroxyl group;
   a C₁-C₄ alkoxy radical; a C₂-C₄ (poly)hydroxyalkoxy radical;
   an amino radical;
   an amino radical substituted by one or two identical or different C₁-C₄ alkyl radicals which optionally carry at least one hydroxyl or amino or C₁-C₄ (mono- or di-)alkylamino or C₁-C₂ alkoxy group, it being possible for the two alkyl radicals, with the nitrogen atom to which they are attached, to form a heterocycle containing 1 to 3 heteroatoms, preferably 1 or 2 heteroatoms, selected from N, O and S, preferably N, the heterocycle containing 5 to 7 ring members, being saturated or unsaturated and aromatic or non-aromatic, and optionally being substituted;
   a an alkylcarbonylamino radical (R′CO—NR—) in which the radical R is a hydrogen atom or a C₁-C₄ alkyl radical and the radical R′ is a C₁-C₂ alkyl radical;
   an aminocarbonyl radical ((R)₂N—CO—) in which the radicals R, which are identical or not, represent a hydrogen atom or a C₁-C₄ alkyl radical;
   an alkylsulphonylamino radical (R′SO₂—NR—) in which the radical R represents a hydrogen atom or a C₁-C₄ alkyl radical and the radical R′ represents a C₁-C₄ alkyl radical or a phenyl radical;
   an aminosulphonyl radical ((R)₂N—SO₂—) in which the radicals R, which are identical or not, represent a hydrogen atom or a C₁-C₄ alkyl radical.

The compounds according to the present invention are termed symmetrical when there exists a plane of symmetry perpendicular to the linker L. In other words, the two formula members either side of the linker are identical. Where the different groups forming part of the structure of the compounds according to the invention are substituted, the skilled person will select them such that the symmetry of the molecule is respected.

The aim of the present invention is to provide direct dyes which do not exhibit the drawbacks of existing direct dyes.

The present invention accordingly provides symmetrical cationic diazo compounds of formula (I) below, their resonance forms, and their acid addition salts and/or their solvates: 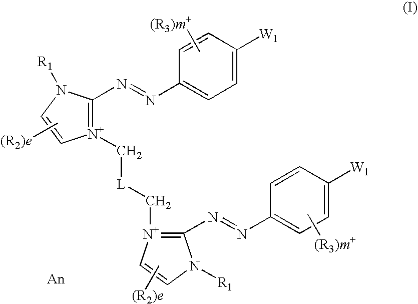
in which formula:

The radicals R₁, which are identical or not, represent:
an optionally substituted C₁-C₄ alkyl radical;
an optionally substituted phenyl radical;
an optionally substituted benzyl radical;
   the radicals R₂, which are identical or not, represent:
an optionally substituted C₁-C₁₆ alkyl radical optionally interrupted by one or more heteroatoms and/or by one or more groups containing at least one heteroatom and selected preferably from oxygen, nitrogen, sulphur, —CO—, —SO₂— or combinations thereof, said alkyl radical being further optionally substituted by one or more groups selected from thio (—SH), C₁-C₄ thioalkyl; C₁-C₄ alkylsulphinyl or C₁-C₄ alkylsulphonyl groups;
a hydroxyl group,
a C₁-C₄ alkoxy group,
a C₂-C₄ (poly)hydroxyalkoxy group;
an alkoxycarbonyl group (RO—CO—) in which R represents a C₁-C₄ alkyl radical,
an alkylcarbonyloxy radical (RCO—O—) in which R represents a C₁-C₄ radical,
an alkylcarbonyl radical (R—CO—) in which R represents a C₁-C₄ alkyl radical,
an amino group,
an amino group substituted by one or two identical or different C₁-C₄ alkyl radicals optionally carrying at least one hydroxyl group, it being possible for the two alkyl radicals optionally to form, with the nitrogen atom to which they are attached, a heterocycle containing 1 to 3 heteroatoms, preferably 1 to 2 heteroatoms, selected from N, O and S, preferably N, and containing 5 to 7 ring members, which is saturated or unsaturated, aromatic or non-aromatic and is optionally substituted;
an alkylcarbonylamino group (RCO—NR′—) in which the radical R represents a C₁-C₄ alkyl radical and the radical R′ represents hydrogen or a C₁-C₄ alkyl radical;
an aminocarbonyl group ((R)₂N—CO—) in which the radicals R independently of one another represent a hydrogen atom or a C₁-C₄ alkyl radical;
a ureido group (N(R)₂—CO—NR′—) in which the radicals R and R′ independently of one another represent a hydrogen atom or a C₁-C₄ alkyl radical;
an aminosulphonyl group ((R)₂N—SO₂—) in which the radicals R independently of one another represent a hydrogen atom or a C₁-C₄ alkyl radical;
an alkylsulphonylamino group (RSO₂—NR′—) in which R represents a C₁-C₄ alkyl radical and R′ represents a hydrogen atom or a C₁-C₄ alkyl radical;
an optionally substituted aryl radical;
an optionally substituted (C₁-C₄ alkyl)aryl radical;
an alkylsulphinyl group (R—SO—) in which R represents a C₁-C₄ alkyl radical;
an alkylsulphonyl group (R—SO₂—) in which R represents a C₁-C₄ alkyl radical;
a nitro group;
a cyano group;
a halogen atom, preferably chlorine or fluorine;
a thio group (HS—);
an alkylthio group (RS—) in which the radical R represents an optionally substituted C₁-C₄ alkyl radical;
when e is 2, the two radicals R₂ may optionally form, with the carbon atoms to which they are attached, a secondary ring, aromatic or non-aromatic, containing 5 or 6 ring members, preferably 6 members, which is optionally substituted by one or more identical or non-identical groups selected from hydroxyl, C₁-C₄ alkyl, C₁-C₄ alkoxy, C₂-C₄ (poly)hydroxyalkoxy, amino, amino substituted by one or two identical or different C₁-C₄ alkyl radicals which optionally carry at least one hydroxyl group;
   e is an integer from 0 to 2; when e is less than 2, the unsubstituted carbon atom(s) of the heterocycle carry a hydrogen atom,
   the radicals R₃, which are identical or not, represent:
an optionally substituted C₁-C₁₆ alkyl radical optionally interrupted by one or more heteroatoms or by one or more groups containing at least one heteroatom and selected preferably from oxygen, nitrogen, sulphur, —CO—, —SO₂— or combinations thereof,
a hydroxyl group,
a C₁-C₄ alkoxy group,
a C₂-C₄ (poly)hydroxyalkoxy group;
an alkoxycarbonyl group (RO—CO—) in which R represents a C₁-C₄ alkyl radical,
an alkylcarbonyloxy radical (RCO—O—) in which R represents a C₁-C₄ alkyl radical;
an alkylcarbonyl radical (R—CO—) in which R represents a C₁-C₄ alkyl radical;
an amino group;
an amino group substituted by one or two identical or different C₁-C₄ alkyl radicals optionally carrying at least one hydroxyl group; it being possible for the two alkyl radicals optionally to form, with the nitrogen atom to which they are attached, a heterocycle containing 1 to 3 heteroatoms, preferably 1 to 2 heteroatoms, selected from N, O and S, preferably N, and containing 5 to 7 ring members, which is saturated or unsaturated, aromatic or non-aromatic and is optionally substituted;
an alkylcarbonylamino group (RCO—NR′—) in which the radical R represents a C₁-C₄ alkyl radical and the radical R′ represents a hydrogen atom or a C₁-C₄ alkyl radical;
an aminocarbonyl group ((R)₂N—CO—) in which the radicals R independently of one another represent a hydrogen atom or a C₁-C₄ alkyl radical;
a ureido group (N(R)₂—CO—NR′—) in which the radicals R and R′ independently of one another represent a hydrogen atom or a C₁-C₄ alkyl radical;
an aminosulphonyl group ((R)₂N—SO₂—) in which the radicals R independently of one another represent a hydrogen atom or a C₁-C₄ alkyl radical;
an alkylsulphonylamino group (RSO₂—NR′—) in which the radicals R and R′ independently of one another represent a hydrogen atom or a C₁-C₄ alkyl radical;
a thio group (HS—);
an alkylthio group (RS—) in which the radical R represents a C₁-C₄ alkyl radical;
an alkylsulphinyl group (R—SO—) in which R represents a C₁-C₄ alkyl radical;
an alkylsulphonyl group (R—SO₂—) in which R represents a C₁-C₄ alkyl radical;
a nitro group;
a cyano group;
a halogen atom, preferably chlorine or fluorine;
when m′ is greater than or equal to 2, two adjacent radicals R₃ may form, with the carbon atoms to which they are attached, a secondary ring, aromatic or non-aromatic, containing 6 ring members, which is optionally substituted by one or more groups selected from the following groups: hydroxyl, C₁-C₄ alkyl, C₁-C₄ alkoxy, C₂-C₄ (poly)hydroxyalkoxy, amino, amino substituted by one or two identical or different C₁-C₄ alkyl radicals which optionally carry at least one hydroxyl group,
   m′ is an integer from 0 to 4; when m′ is less than 4, the unsubstituted carbon atom(s) of the heterocycle carry a hydrogen atom;
   W₁ radicals, which are identical, represent:
a hydrogen atom,
a halogen atom selected from bromine, chlorine and fluorine, preferably chlorine and fluorine,
an —NR₅R₆, OR₇, —NR₄—Ph—NR₅R₆, —NR₄—Ph—OR₇, —O—Ph—OR₇ or —O—Ph—NR₅R₆ group; where:
   R₄ and R₇, which are identical or not, represent a hydrogen atom, an optionally substituted C₁-C₂₀, preferably C₁-C₁₆, alkyl radical, an optionally substituted C₁-C₃ aralkyl radical or an optionally substituted phenyl radical;
   R₅ and R₆, which are identical or not, represent a hydrogen atom, an optionally substituted C₁-C₂₀, preferably C₁-C₁₆, alkyl radical, an optionally substituted phenyl radical, an optionally substituted C₁-C₃ aralkyl radical or an alkylcarbonyl radical (R—CO—) in which R is a C₁-C₄ alkyl radical;
   R₅ and R₆ may optionally form, with the nitrogen atom to which they are attached, a heterocycle containing 1 to 3 heteroatoms, preferably 1 or 2 heteroatoms, selected from N, O and S, preferably N, and containing 5 to 7 ring members, which is saturated or unsaturated, aromatic or non-aromatic and is optionally substituted;
   R₅ and R₆, independently of one another, may form, with the carbon atom of the aromatic ring adjacent to that to which —NR₅R₆ is attached, a 5- or 6-membered saturated heterocycle;
   Ph represents an optionally substituted phenyl radical;
      L, a cationic linker connecting the two identical azo chromophores, represents a C₂-C₄₀ alkyl radical which carries at least one cationic charge and is optionally substituted and/or optionally interrupted by one or more saturated or unsaturated, aromatic or non-aromatic, identical or different (hetero)cycles containing 3 to 7 ring members and/or optionally interrupted by one or more heteroatoms or groups containing at least one heteroatom, or combinations thereof, such as, for example, oxygen, nitrogen, sulphur, —CO—, —SO₂— or combinations thereof, the linker L containing no azo, nitro, nitroso or peroxo bond; with the proviso that the linker L carries at least one cationic charge; the electroneutrality of the compound of formula (I) being ensured by one or more identical or non-identical, cosmetically acceptable anions An.

The present invention also provides dyeing compositions comprising such compounds, or their addition salts with an acid, as direct dyes in a medium appropriate for the dyeing of keratin fibres.

It further provides a method of colouring keratin fibres which consists in contacting a composition according to the invention with said fibres, which are dry or wet, for a time sufficient to give the desired effect.

It provides, finally, a device having a plurality of compartments and containing in a first compartment the composition according to the invention and in a second compartment an oxidizing composition.

It has been found that the compounds of formula (I) as defined above exhibit effective resistance to external agents such as, in particular, shampoos, and do so even when the keratin fibre is sensitized. Furthermore, these compounds exhibit improved dyeing properties, such as the chromaticity, the colouring power, and a low selectivity, which is to say that the compounds of the invention allow colorations to be obtained which are more uniform between the end and the root of the hair.

Other characteristics and advantages of the invention, however, will appear more clearly from reading the description and the examples which will be presented.

In the text below, and in the absence of any indication otherwise, the end-points delimiting a range of values are included in that range.

As indicated above, the invention first provides compounds corresponding to the aforementioned formula (I).

Preferably the compounds of formula (I) according to the present invention are such that the identical radicals R₁ represent:
a C₁-C₄ alkyl radical which is optionally substituted by one or more radicals selected from hydroxyl, C₁-C₂ alkoxy, amino and C₁-C₂ (di)alkylamino radicals;
an optionally substituted benzyl radical.

According to one particularly preferred embodiment, the identical groups R₁ represent a methyl, ethyl, 2-hydroxethyl, 2-methoxyethyl or benzyl radical.

The radicals R₂, which are identical or not, preferentially represent:
a halogen atom selected from chlorine and fluorine;
a C₁-C₄ alkyl radical optionally substituted by one or more identical or different radicals selected from hydroxyl, C₁-C₂ alkoxy, C₂-C₄ (poly)hydroxyalkoxy, amino, C₁-C₂ (di)alkylamino, thio (—SH), C₁-C₄ alkylsulphinyl, C₁-C₄ alkylsulphonyl and C₁-C₄ thioalkyl radicals;
a phenyl radical optionally substituted by one or more identical or different radicals selected from hydroxyl, C₁-C₂ alkoxy, C₂-C₄ (poly)hydroxyalkoxy, amino and C₁-C₂ (di)alkylamino radicals or a halogen atom such as chlorine or fluorine;
a C₁-C₄ alkoxy radical;
a C₁-C₄ alkylsulphonylamino radical;
a C₂-C₄ (poly)hydroxyalkoxy radical;
an amino radical;
a C₁-C₂ (di)alkylamino radical;
a C₂-C₄ (poly)hydroxyalkylamino radical;
an alkylsulphonylamino radical (RSO₂N—) in which the radical R represents C₁-C₄ alkyl radical;
an aminosulphonyl radical ((R)₂NSO₂—) in which the radicals R independently of one another represent a hydrogen atom or a C₁-C₄ alkyl radical; an alkylthio radical (RS—) in which the radical R represents a C₁-C₄ alkyl radical; an alkylsulphinyl radical (RSO—) in which the radical R represents a C₁-C₄ alkyl radical; an alkylsulphonyl radical (R—SO₂—) in which the radical R represents a C₁-C₄ alkyl radical;
an alkylcarbonylamino radical (RCONR′—) in which the radical R represents a hydrogen atom or a C₁-C₄ alkyl radical and the radical R′ represents a hydrogen atom or a C₁-C₄ alkyl radical.

According to one particularly preferred embodiment, the identical or non-identical radicals R₂ represent preferably a methyl, ethyl, 2-hydroxyethyl, 2-methoxyethyl, methylsulphonyl (CH₃SO₂—), methylcarbonylamino (CH₃CON H—), hydroxyl, amino, methylamino, dimethylamino, 2-hydroxyethylamino, methoxy, ethoxy or phenyl radical.

According to a second preferred version, the two radicals R₂ may optionally form, with the carbon atoms to which they are attached, a secondary, 6-membered aromatic ring optionally substituted by one or more identical or different groups selected from hydroxyl, C₁-C₄ alkyl, C₁-C₄ alkoxy, amino, and (di)(C₁-C₄)-alkylamino which optionally carry at least one hydroxyl or methylcarbonylamino group.

In accordance with this second version, the two radicals R₂ may optionally form, with the carbon atoms to which they are attached, a secondary, 6-membered aromatic ring optionally substituted by one or more hydroxyl, methoxy, ethoxy, amino, acylamino, 2-hydroxyethylamino, dimethylamino, (di)-2-hydroxyethylamino or methylcarbonylamino substituents.

With regard more particularly to the radicals R₃, these radicals, which are identical or different, represent more particularly:
an optionally substituted C₁-C₁₆, preferably C₁-C₈, alkyl radical;
a halogen atom such as chlorine or fluorine;
a hydroxyl group;
a C₁-C₂ alkoxy radical;
a C₂-C₄ (poly)hydroxyalkoxy radical;
an amino radical;
an amino radical substituted by one or two identical or different C₁-C₄ alkyl radicals which optionally carry at least one hydroxyl group or at least one C₁-C₄ alkoxy radical, it being possible for the two alkyl radicals to form, with the nitrogen to which they are attached, a heterocycle containing 1 to 3 heteroatoms, preferably 1 or 2 heteroatoms, selected from N, O and S, preferably N, the heterocycle containing 5 to 7 ring members, being saturated or unsaturated, aromatic or non-aromatic, and being optionally substituted;
an alkylcarbonylamino radical (RCO—NR′—) in which the radical R represents a C₁-C₄ alkyl radical and the radical R′ represents a hydrogen or a C₁-C₄ radical;
an alkylsulphonylamino radical (R′SO₂—NR—) in which the radical R represents a hydrogen atom or a C₁-C₄ alkyl radical and the radical R′ represents a C₁-C₄ alkyl radical;
an aminosulphonyl radical ((R)₂N—SO₂—) in which the radicals R, which are identical or not, represent a hydrogen atom or a C₁-C₄ alkyl radical;
an alkylthio radical (RS—) in which the radical R represents a C₁-C₄ alkyl radical;
an alkylsulphonyl radical (R—SO₂—) in which the radical R represents a C₁-C₄ alkyl radical.

More preferably said radicals R₃, which are identical or different, represent:
a C₁-C₄ alkyl radical optionally substituted by one or more radicals selected from hydroxyl radicals, C₁-C₂ alkylcarbonylamino radicals, amino radicals substituted by two identical or different C₁-C₂ alkyl radicals which optionally carry at least one hydroxyl group or a C₁-C₂ alkoxy radical, it being possible for these two alkyl radicals optionally to form, with the nitrogen atom to which they are attached, a 5- or 6-membered heterocycle which is saturated or unsaturated and is optionally aromatic, selected preferably from pyrrolidine, piperazine, homopiperazine, pyrrole, imidazole and pyrazole;
a C₂-C₄ hydroxyalkoxy radical;
a halogen selected from chlorine and fluorine;
an amino radical;
an amino radical substituted by one or two identical or different C₁-C₂ alkyl radicals which optionally carry at least one hydroxyl group;
a methylcarbonylamino radical;
a methylsulphonylamino radical;
a hydroxyl radical;
a C₁-C₂ alkoxy radical;
a methylsulphonyl radical.

According to this version, the radicals R₃, independently of one another, represent preferably:
a methyl, ethyl, propyl, 2-hydroxyethyl, methoxy, ethoxy, 2-hydroxyethyloxy, 3-hydroxypropyloxy or 2-methoxyethyl radical;
a methylsulphonylamino radical;
an amino, methylamino, dimethylamino or 2-hydroxyethylamino radical;
a methylcarbonylamino radical;
a hydroxyl radical;
a chlorine atom;
a methylsulphonyl radical.

According to a second preferred version, when the coefficient m′ is greater than or equal to 2, two adjacent radicals R₃ may form, with the carbon atoms to which they are attached, a secondary, 6-membered aromatic ring optionally substituted by one or more groups selected from hydroxyl groups, —NR₄—Ph, —NR₄—Ph—NR₅R₆ and —NR₄—Ph—OR₇ groups, C₁-C₄ alkyl groups, C₁-C₄ alkoxy groups, C₂-C₄ (poly)hydroxyalkoxy groups, C₁-C₄ alkylcarbonylamino groups, amino groups, and (di)alkylamino groups in which the C₁-C₄ alkyl radical(s) optionally carry at least one hydroxyl group.

According to this second version, and even more advantageously, two adjacent radicals R₃ may form, with the carbon atoms to which they are attached, a secondary, 6-membered aromatic ring which is optionally substituted by one or more groups selected from hydroxyl, methoxy, ethoxy, 2-hydroxyethyloxy, amino, methylcarbonylamino, (di)-2-hydroxyethylamino, —NH—Ph, —NH—Ph—NH₂, —NH—Ph—NHCOCH₃, —NH—Ph—OH and —NH—Ph—OCH₃ groups.

With regard to the radicals R₄ and R₇, these radicals represent:
a hydrogen atom;
a C₁-C₆ alkyl radical which is optionally substituted by one or more identical or different groups selected from hydroxyl and C₁-C₂ alkoxy;
an aryl or arylalkyl radical, such as phenyl or benzyl, the aryl moiety being optionally substituted by one or more identical or different groups selected from chlorine, amino, hydroxyl, C₁-C₂ alkoxy, amino which is mono- or disubstituted by two identical or different radicals selected from C₁-C₄ alkyl radicals which optionally carry at least one hydroxyl group.

In accordance with one preferred embodiment of the invention, the radicals R₄ and R₇ represent:
a hydrogen atom;
an optionally substituted C₁-C₃ alkyl radical, such as methyl, ethyl, 2-hydroxyethyl or 2-methoxyethyl;
a phenyl radical which is optionally substituted by one or more radicals selected from hydroxyl radicals, C₁-C₂ alkoxy radicals, amino radicals, and amino radicals substituted by one or more C₁-C₄ groups which optionally carry at least one hydroxyl group;
   preferably, the radicals R₄ and R₇ represent:
a hydrogen atom;
a methyl, ethyl or 2-hydroxyethyl radical;
a phenyl radical which is optionally substituted by a hydroxyl, methoxy, amino, (di)methylamino or (di)(2-hydroxyethyl)amino radical.

With regard to the radicals R₅ and R₆, independently of one another, these radicals represent more particularly:
a hydrogen atom;
an alkylcarbonyl radical (R—CO—) in which R represents an optionally substituted C₁-C₄ alkyl radical,
a C₁-C₆ alkyl radical which is optionally substituted, preferably by at least one hydroxyl group, at least one C₁-C₂ alkoxy group, at least one amino group, at least one C₁-C₄ (di)alkylamino group; the alkyl radical may further be substituted by one or more identical or different groups selected from C₁-C₄ alkylsulphonyl, C₁-C₄ alkylsulphinyl and C₁-C₄ alkylcarbonyl,
an aryl or arylalkyl radical, such as phenyl or benzyl, the aryl moiety being optionally substituted by one or more identical or different groups selected from chlorine, amino, hydroxyl, C₁-C₄ alkoxy, amino which is mono- or disubstituted by two identical or different radicals selected from C₁-C₄ alkyl radicals which optionally carry at least one hydroxyl group.

In accordance with one preferred embodiment of the invention, the radicals R₅ and R₆, which are identical or different, represent advantageously:
a hydrogen atom;
a methylcarbonyl, ethylcarbonyl or propylcarbonyl radical;
an optionally substituted C₁-C₃ alkyl radical, such as methyl, ethyl, 2-hydroxyethyl or 2-methoxyethyl;
a phenyl radical which is optionally substituted by one or more radicals selected from hydroxyl radicals, C₁-C₂ alkoxy radicals, amino radicals, and amino radicals substituted by one or more C₁-C₄ groups which optionally carry at least one hydroxyl group.

More preferably still, the radicals R₅ and R₆, which are identical or different, represent:
a hydrogen atom;
a methyl, ethyl or 2-hydroxyethyl radical;
a methylcarbonyl, ethylcarbonyl or propylcarbonyl radical;
a phenyl radical which is optionally substituted by a hydroxyl, methoxy, amino, (di)methylamino or (di)(2-hydroxyethyl)amino radical.

It should be noted that, according to one particular embodiment of the invention, the radicals R₅ and R₆ form, together with the nitrogen atom to which each is attached, a heterocycle containing 1 to 3 heteroatoms, preferably 1 or 2 heteroatoms, selected from N, O and S, preferably N, and containing 5 to 7 ring members, which is saturated or unsaturated, aromatic or non-aromatic, and is optionally substituted.

Advantageously, the heterocycle containing 5 to 7 ring members is selected from the following heterocycles: piperidine, 2-(2-hydroxyethylpiperidine), 4-(aminomethyl)piperidine, 4-(2-hydroxyethyl)piperidine, 4-(dimethylamino)piperidine, piperazine, 1-methylpiperazine, 1-(2-hydroxyethyl)piperazine, 1-(2-aminoethyl)piperazine, 1-hydroxyethylethoxypiperazine, homopiperazine, 1-methyl-1,4-perhydrodiazepine, pyrrole, 1,4-dimethylpyrrole, 1-methyl-4-ethylpyrrole, 1-methyl-4-propylpyrrole.

Preferably, the heterocycle containing 5 to 7 ring members represents a heterocycle of piperidine, piperazine, homopiperazine, pyrrole, imidazole or pyrazole type which is optionally substituted by one or more methyl, hydroxyl, amino and/or (di)methylamino radicals.

According to a third variant, the radicals R₅ and R₆ represent alkyl radicals which, independently of one another, form, with the carbon atom of the aromatic ring optionally substituted by a hydroxyl and adjacent to that to which —NR₅R₆ is attached, a 5- or 6-membered saturated heterocycle.

For example, the group —NR₅R₆ with the aromatic nucleus optionally substituted by a hydroxyl may correspond to the following compounds:

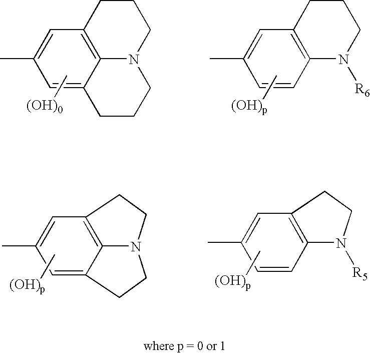

The linker L advantageously represents a C₂-C₂₀ alkyl radical:
1—interrupted by at least one group corresponding to the following formulae: 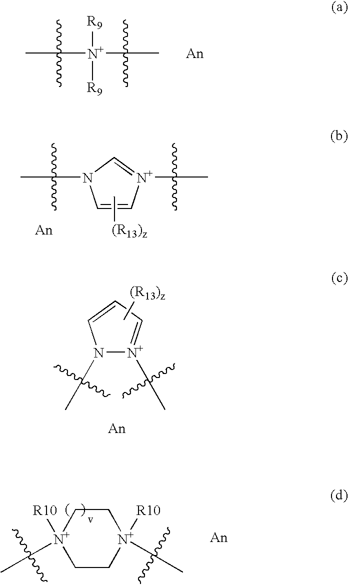
   in which:
   R₉ and R₁₀ independently of one another represent a C₁-C₈ alkyl radical; a C₁-C₆ monohydroxyalkyl radical; a C₂-C₆ polyhydroxyalkyl radical; a C₁-C₆ alkoxy-C₁-C₆ alkyl radical; an aryl radical such as phenyl which is optionally substituted; an arylalkyl radical such as benzyl which is optionally substituted; a C₁-C₆ aminoalkyl radical; a C₁-C₆ aminoalkyl radical whose amine is substituted by one or more identical or different C₁-C₄ alkyl radicals; or a C₁-C₆ alkylsulphonyl radical,
   two radicals R₉ may together form, with the nitrogen atom to which they are attached, a saturated or unsaturated ring which is optionally substituted and has 5, 6 or 7 ring members,

R₁₃ radicals, which are identical or different, represent a halogen atom selected from bromine, chlorine and fluorine, a C₁-C₆ alkyl radical, a C₁-C₆ monohydroxyalkyl radical, a C₂-C₆ polyhydroxyalkyl radical, a C₁-C₆ alkoxy radical, a C₁-C₄ (di)alkylamino radical, a hydroxycarbonyl radical, a C₁-C₆ alkylcarbonyl radical, a C₁-C₆ thioalkyl radical, a C₁-C₆ alkylthio radical, an amino radical (di)substituted by a (C₁-C₆)-alkyl radical, a C₁-C₆ alkylsulphonyl radical, an optionally substituted benzyl radical, or a phenyl radical which is optionally substituted by one or more radicals selected from methyl, hydroxyl, amino and methoxy radicals,
An is an organic or inorganic anion or anion mixture,
z is an integer between 1 and 3; if z is <3, the unsubstituted carbon atoms carry a hydrogen atom;
v is an integer 1 or 2, preferably 1,
2—optionally interrupted by one or more heteroatoms or groups containing at least one heteroatom, or combinations thereof, such as, for example, oxygen, nitrogen, sulphur, a —CO— group or a —SO₂— group, with the proviso that there is no nitro, nitroso or peroxo bond or group in the linker L;
3—and optionally substituted by one or more radicals selected from hydroxyl radicals, C₁-C₂ alkoxy radicals, C₂-C₄ (poly)hydroxyalkoxy radicals, amino radicals substituted by one or more linear or branched C₁-C₂ alkyl groups which optionally carry at least one hydroxyl group.

According to one particular embodiment of formulae (a) and (d), R₉ and R₁₀, separately, are preferably selected from a C₁-C₆ alkyl radical, a C₁-C₄ monohydroxyalkyl radical, a C₂-C₄ polyhydroxyalkyl radical, a C₁-C₆ alkoxy-C₂-C₄ alkyl radical and a C₂-C₆ dimethylaminoalkyl radical.

With further preference, R₉ and R₁₀ separately represent a methyl, ethyl or 2-hydroxyethyl radical.

According to one particular embodiment of formulae (b) and (c), R₁₃ represents a halogen atom selected from chlorine and fluorine or a C₁-C₈ alkyl radical, a C₁-C₄ monohydroxyalkyl radical, a C₁-C₄ alkoxy radical, a hydroxycarbonyl radical, a C₁-C₆ alkylthio radical or an amino radical disubstituted by a C₁-C₄ alkyl radical.

According to one even more particular embodiment of formulae (b) and (c), R₁₃ represents a chlorine atom, a methyl, an ethyl, a 2-hydroxyethyl, a methoxy, a hydroxycarbonyl or a dimethylamino. According to another even more particular embodiment of formulae (b) and (c), z is 0.

In the formula (I) An represents an organic or inorganic anion or anion mixture allowing the charge or charges on the compounds of formula (I) to be balanced, and selected for example from a halide such as chloride, bromide, fluoride or iodide; a hydroxide; a sulphate; a hydrogensulphate; an alkylsulphate for which the linear or branched alkyl moiety is C₁-C₆, such as the methylsulphate or ethylsulphate ion; carbonates and hydrogencarbonates; salts of carboxylic acids, such as formate, acetate, citrate, tartrate and oxalate; alkylsulphonates for which the linear or branched alkyl moiety is C₁-C₆, such as the methylsulphonate ion; arylsulphonates for which the aryl moiety, preferably phenyl, is optionally substituted by one or more C₁-C₄ radicals, such as 4-tolylsulphonate, for example; and alkylsulphonyls such as mesylate.

Furthermore, the acid addition salts of the compounds of formula (I) may be, by way of example, the addition salts with an organic or inorganic acid such as hydrochloric acid, hydrobromic acid, sulphuric acid or (alkyl- or phenyl-)sulphonic acids such as p-toluenesulphonic acid or methylsulphonic acid.

Finally, the solvates of compounds of formula (I) more especially represent the hydrates of such compounds or the combination of compounds of formula (I) with a linear or branched C₁-C₄ alcohol such as methanol, ethanol, isopropanol or n-propanol.

In accordance with one preferred embodiment of the invention, the compounds correspond to formula (I′) below, and also to its resonance forms and/or its acid addition salts and/or its solvates:

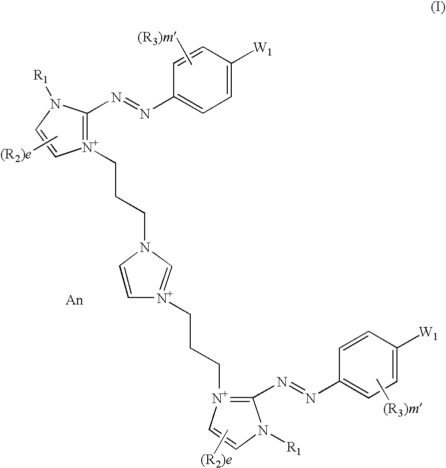

The groups W₁, the radicals R₁, R₂, R₃, the coefficients e and m′ being defined as above.

In accordance with one preferred embodiment of the invention, the compounds correspond to the formula below, and also to its resonance forms, its acid addition salts and/or its solvates: 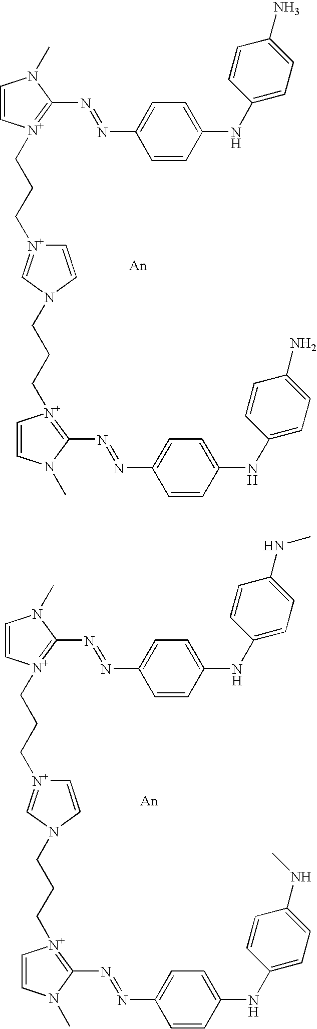 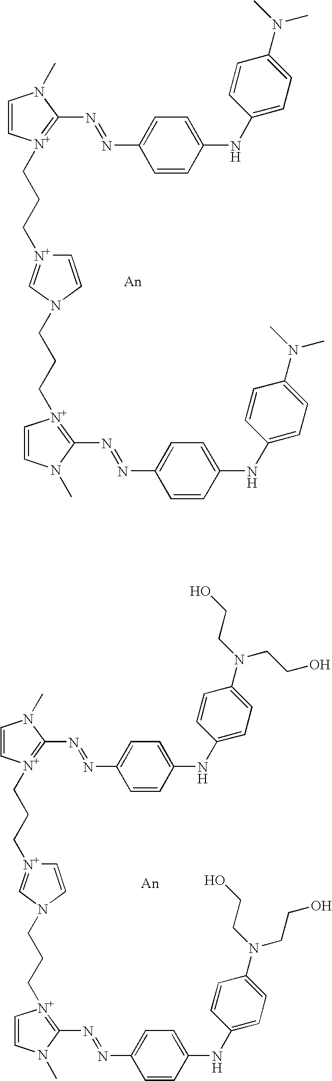 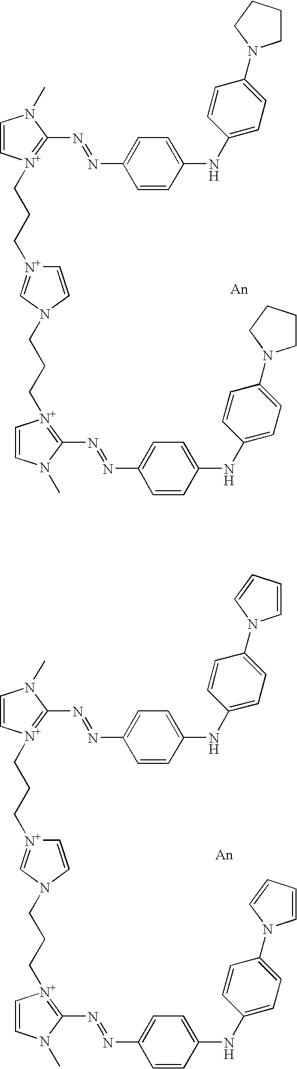 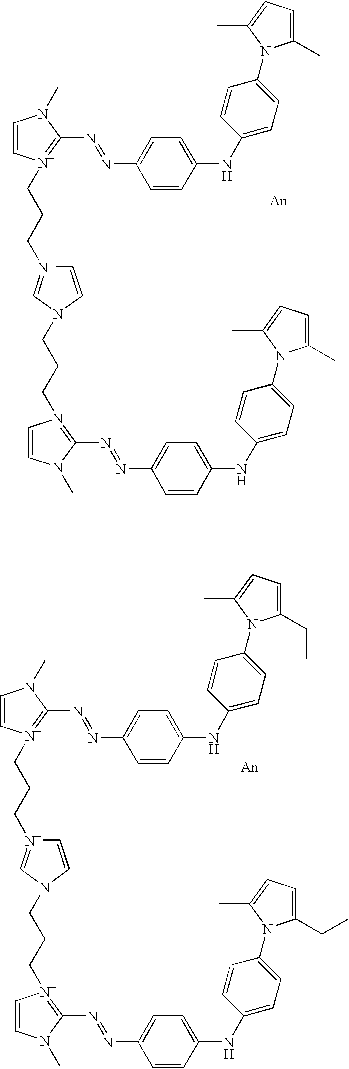 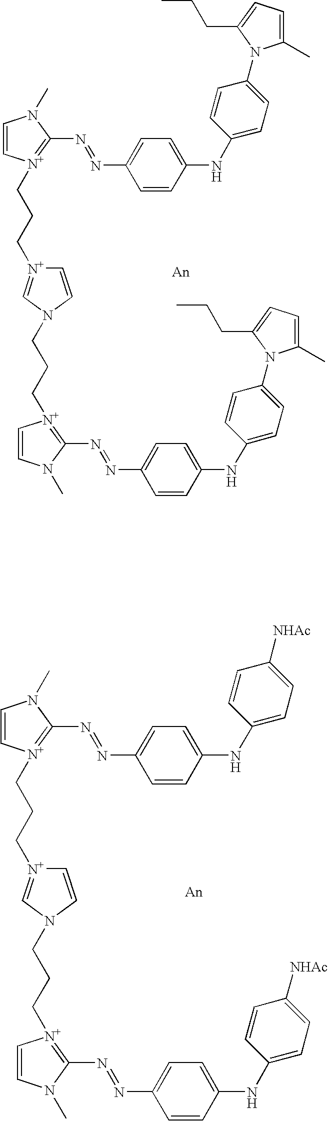 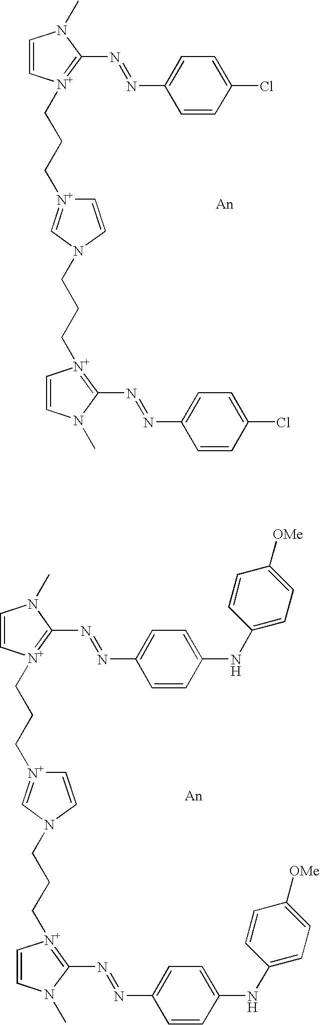 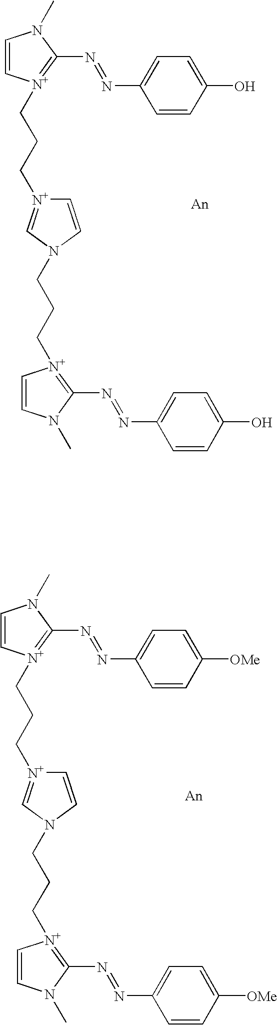 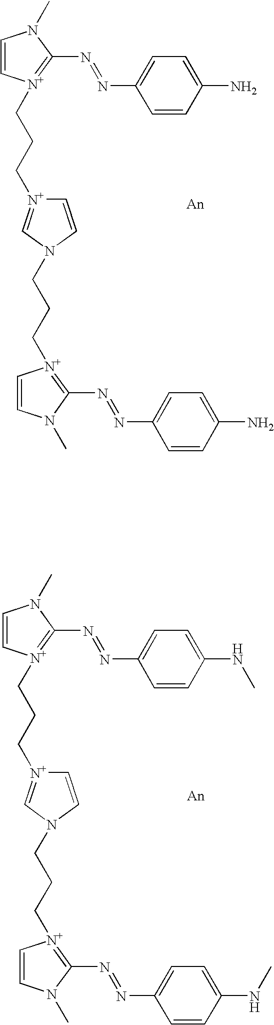 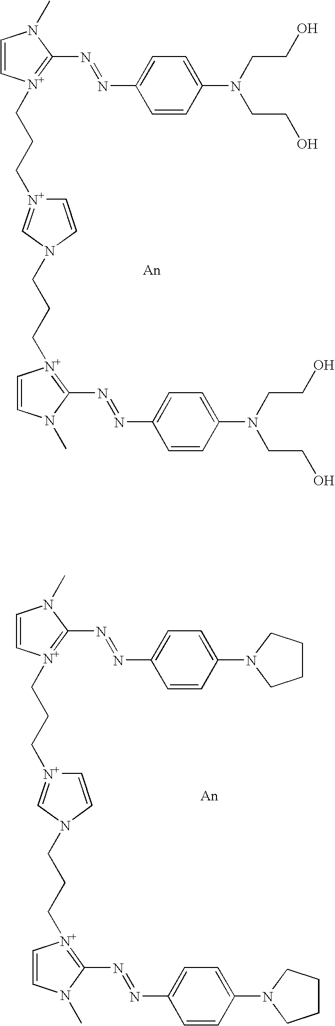 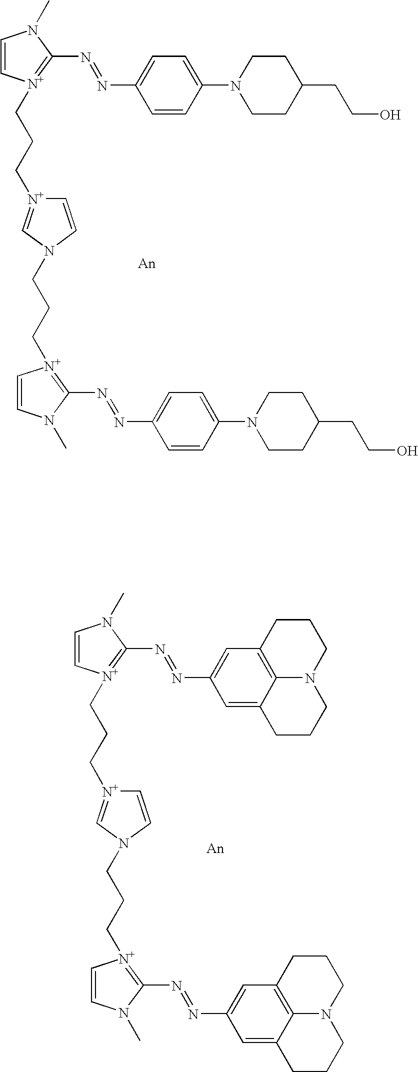 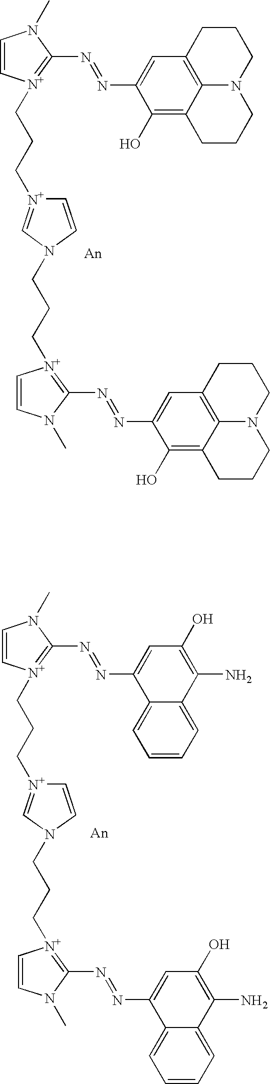 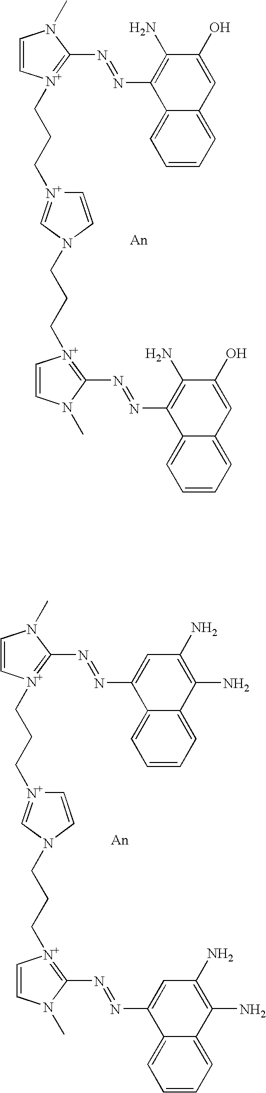 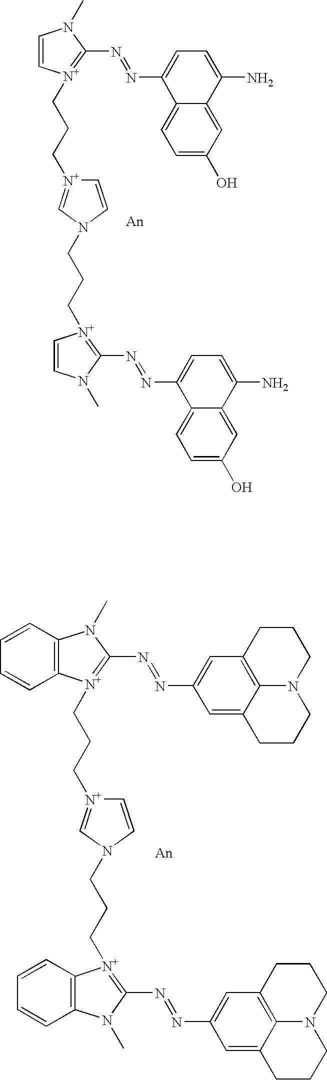 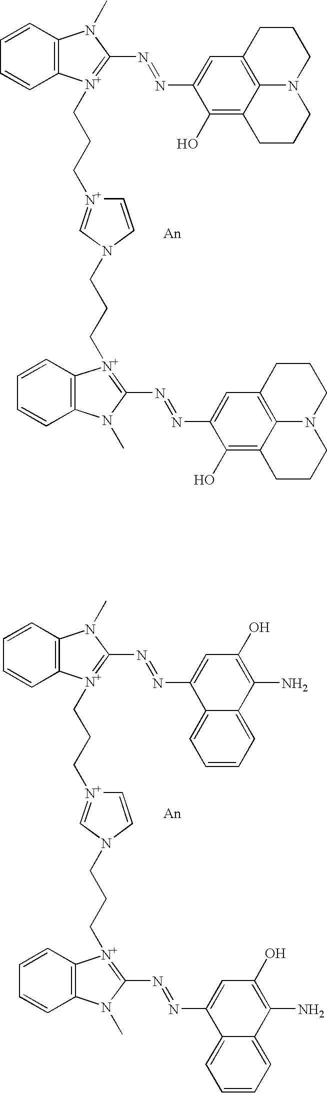 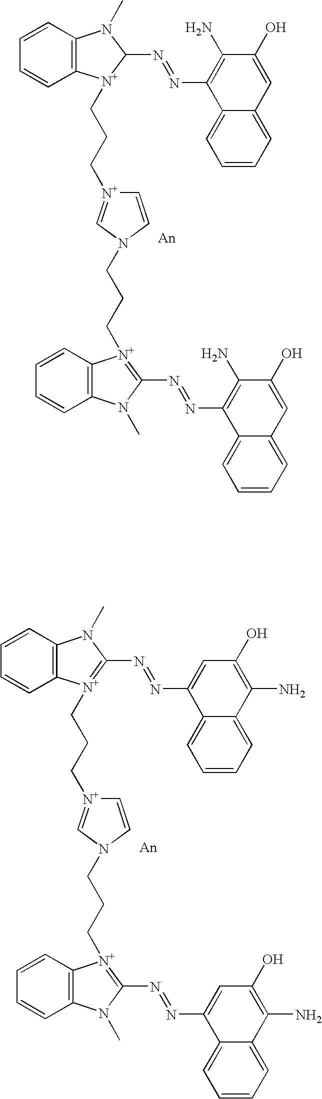 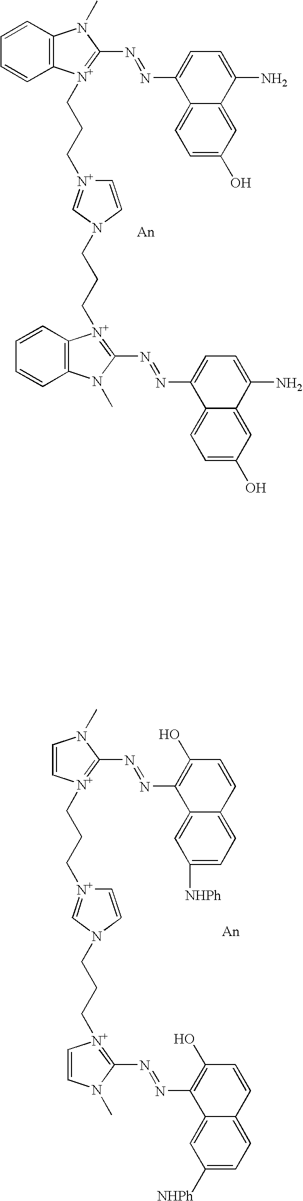 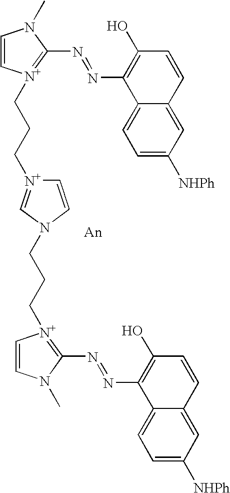
compounds corresponding to the monoazo species may in particular be obtained from preparation processes described, for example, in the documents U.S. Pat. No. 5,708,151, J. Chem. Res., Synop. (1998), (10), 648-9, U.S. Pat. No. 3,151,106, U.S. Pat. No. 5,852,179, Heterocycles, 1987, 26 (2) 313-7, Synth. Commun. 1999, 29 (13), 2271-6, Tetrahedron, 1983, 39 (7), 1091-1101. As for the diazo compounds, reference may be made to European patent application EP 1377263 for a synthesis description.

The present invention further provides a dyeing composition comprising at least one compound of formula (I), or its acid addition salts, as direct dye in a medium appropriate for the dyeing of keratin fibres.

The total concentration of compound(s) of formula (I) may vary between 0.001% and 20% by weight relative to the total weight of the dyeing composition, more particularly between 0.01% and 10% by weight and preferably between 0.05% and 5% by weight.

The dyeing composition according to the invention may also comprise an oxidation base. This oxidation base may be selected from the oxidation bases conventionally used in oxidation dyeing, for example para-phenylenediamines, bis(phenyl)alkylenediamines, para-aminophenols, ortho-aminophenols and heterocyclic bases.

Among the para-phenylenediamines that may be mentioned more particularly are, for example, para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxy-ethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-amino-phenylpyrrolidine, 2-thienyl-para-phenylenediamine, 2-β-hydroxyethylamino-5-aminotoluene, and the addition salts thereof with an acid.

Among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid, are particularly preferred.

Among the bis(phenyl)alkylenediamines that may be mentioned, for example, are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-amino-phenyl)tetramethylenediamine, N,N′-bis(β-hydroxyethyl)-N,N′-bis(4-aminophenyl)tetramethylenediamine, N,N′-bis(4-methyl-aminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof with an acid.

Among the para-aminophenols that may be mentioned, for example, are para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetamido-2-aminophenol, and the addition salts thereof with an acid.

Among the heterocyclic bases that may be mentioned, for example, are pyridine derivatives, pyrimidine derivatives and pyrazole derivatives.

Among the pyridine derivatives that may be mentioned are the compounds described, for example, in patents GB 1 026 978 and GB 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diamino-pyridine, and the addition salts thereof with an acid.

Among the pyrimidine derivatives that may be mentioned are the compounds described, for example, in patents DE 2 359 399; JP 88-169 571; JP 05-163 124; EP 0 770 375 or patent application WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those mentioned in patent application FR-A-2 750 048 and among which mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo-[1,5-a]pyrimidine-3,7-diamine, 2,5,N7,N7-tetramethylpyrazolo[1,5-a]pyrimidine-3,7-diamine and 3-amino-5-methyl-7-imidazolylpropylaminopyrazolo[1,5-a]pyrimidine, and the addition salts thereof with an acid and the tautomeric forms thereof, when a tautomeric equilibrium exists.

Among the pyrazole derivatives that may be mentioned are the compounds described in patents DE 3 843 892 and DE 4 133 957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof with an acid.

The dyeing composition according to the invention may also contain one or more couplers conventionally used for dyeing keratin fibres. Among these couplers, mention may be made especially of meta-phenylenediamines, meta-aminophenols, meta-diphenols, naphthalenic couplers and heterocyclic couplers.

Examples that may be mentioned include 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureido-aniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene and 2,6-bis(β-hydroxy-ethylamino)toluene and the addition salts thereof with an acid.

In the dyeing composition of the present invention the oxidation base or bases are present in a total amount of preferably between 0.001% to 10% by weight of the total weight of the dyeing composition, and more preferably of 0.005% to 6% by weight.

The coupler or couplers are generally present in a total amount of between 0.001% and 10% by weight of the total weight of the dyeing composition, and more preferably of 0.005% to 6% by weight.

In general, the acid addition salts that may be used in the context of the dyeing compositions of the invention for the oxidation bases and couplers are selected especially from those listed in the context of the definition of the compounds of formula (I).

The composition according to the invention may optionally comprise at least one additional direct dye other than the compounds of formula (I). This dye may be selected from cationic and nonionic species.

Non-limiting examples that may be mentioned include nitrobenzene dyes, azo, azomethine, methine, tetraazapentamethine, anthraquinone, naphthoquinone, benzoquinone, phenothiazine, indigoid, xanthene, phenanthridine and phthalocyanine dyes, dyes derived from triarylmethane, and natural dyes, alone or as mixtures.

It may be selected, for example, from the following red or orange nitrobenzene dyes:
1-hydroxy-3-nitro-4-N-(γ-hydroxypropyl)aminobenzene,
N-(β-hydroxyethyl)amino-3-nitro-4-aminobenzene,
1-amino-3-methyl-4-N-(β-hydroxyethyl)amino-6-nitrobenzene,
1-hydroxy-3-nitro-4-N-(β-hydroxyethyl)aminobenzene,
1,4-diamino-2-nitrobenzene,
1-amino-2-nitro-4-methylaminobenzene,
N-(β-hydroxyethyl)-2-nitro-para-phenylenediamine,
1-amino-2-nitro-4-(β-hydroxyethyl)amino-5-chlorobenzene,
2-nitro-4-aminodiphenylamine,
1-amino-3-nitro-6-hydroxybenzene,
1-(β-aminoethyl)amino-2-nitro-4-(β-hydroxyethyloxy)benzene,
1-(β,γ-dihydroxypropyl)oxy-3-nitro-4-(β-hydroxyethyl)aminobenzene,
1-hydroxy-3-nitro-4-aminobenzene,
1-hydroxy-2-amino-4,6-dinitrobenzene,
1-methoxy-3-nitro-4-(β-hydroxyethyl)aminobenzene,
2-nitro-4′-hydroxydiphenylamine, and
1-amino-2-nitro-4-hydroxy-5-methylbenzene.

The additional direct dye may also be selected from yellow and green-yellow nitrobenzene direct dyes; mention may be made, for example, of the compounds selected from:
1-β-hydroxyethyloxy-3-methylamino-4-nitrobenzene,
1-methylamino-2-nitro-5-(β,γ-dihydroxypropyl)oxybenzene,
1-(β-hydroxyethyl)amino-2-methoxy-4-nitrobenzene,
1-(β-aminoethyl)amino-2-nitro-5-methoxybenzene,
1,3-di(β-hydroxyethyl)amino-4-nitro-6-chlorobenzene,
1-amino-2-nitro-6-methylbenzene,
1-(β-hydroxyethyl)amino-2-hydroxy-4-nitrobenzene,
N-(β-hydroxyethyl)-2-nitro-4-trifluoromethylaniline,
4-(β-hydroxyethyl)amino-3-nitrobenzenesulphonic acid,
4-ethylamino-3-nitrobenzoic acid,
4-(β-hydroxyethyl)amino-3-nitrochlorobenzene,
4-(β-hydroxyethyl)amino-3-nitromethylbenzene,
4-(β,γ-dihydroxypropyl)amino-3-nitrotrifluoromethylbenzene,
1-(β-ureidoethyl)amino-4-nitrobenzene,
1,3-diamino-4-nitrobenzene,
1-hydroxy-2-amino-5-nitrobenzene,
1-amino-2-[tris(hydroxymethyl)methyl]amino-5-nitrobenzene,
1-(β-hydroxyethyl)amino-2-nitrobenzene, and
4-(β-hydroxyethyl)amino-3-nitrobenzamide.

Mention may also be made of blue or violet nitrobenzene direct dyes; for instance:
1-(β-hydroxyethyl)amino-4-N,N-bis(β-hydroxyethyl)amino-2-nitrobenzene,
1-(γ-hydroxypropyl)amino-4-N,N-bis(β-hydroxyethyl)amino-2-nitrobenzene,
1-(β-hydroxyethyl)amino-4-(N-methyl-N-β-hydroxyethyl)amino-2-nitrobenzene,
1-(β-hydroxyethyl)amino-4-(N-ethyl-N-β-hydroxyethyl)amino-2-nitrobenzene,
1-(β,γ-dihydroxypropyl)amino-4-(N-ethyl-N-β-hydroxyethyl)amino-2-nitrobenzene,
2-nitro-para-phenylenediamines having the following formula: 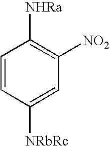
   in which:
   Rb represents a C₁-C₄ alkyl radical or a β-hydroxyethyl, β-hydroxypropyl or γ-hydroxypropyl radical;
   Ra and Rc, which may be identical or different, represent a β-hydroxyethyl, β-hydroxypropyl, γ-hydroxypropyl or β,γ-dihydroxypropyl radical, at least one of the radicals Rb, Rc or Ra representing a γ-hydroxypropyl radical and Rb and Rc not being able to denote simultaneously a β-hydroxyethyl radical when Rb is a γ-hydroxypropyl radical, such as those described in French patent FR 2 692 572.

Among the azo direct dyes that may be used according to the invention, mention may be made of the cationic azo dyes described in patent applications WO 95/15144, WO 95/01772 and EP 714954, FR 2 822 696, FR 2 825 702, FR 2 825 625, FR 2 822 698, FR 2 822 693, FR 2 822 694, FR 2 829 926, FR 2 807 650, WO 02/078660, WO 02/100834, WO 02/100369 and FR 2 844 269.

Among these compounds, mention may be made very particularly of the following dyes:
1,3-dimethyl-2-[[4-dimethyl)phenyl]azo]-1H-imidazolium chloride,
1,3-dimethyl-2-[(4-aminophenyl)azo]-1H-imidazolium chloride,
1-methyl-4-[(methylphenylhydrazono)methyl]pyridinium methylsulphate.

Among the azo direct dyes that may also be mentioned are the following dyes described in the Colour Index International 3rd edition:

Disperse Red 17

Acid Yellow 9

Acid Black 1

Basic Red 22

Basic Red 76

Basic Yellow 57

Basic Brown 16

Acid Yellow 36

Acid Orange 7

Acid Red 33

Acid Red 35

Basic Brown 17

Acid Yellow 23

Acid Orange 24

Disperse Black 9.

Mention may also be made of 1-(4′-aminodiphenylazo)-2-methyl-4-[bis(β-hydroxyethyl)amino]benzene and 4-hydroxy-3-(2-methoxyphenylazo)-1-naphthalenesulphonic acid.

Among the quinone direct dyes that may be mentioned are the following dyes:

Disperse Red 15

Solvent Violet 13

Acid Violet 43

Disperse Violet 1

Disperse Violet 4

Disperse Blue 1

Disperse Violet 8

Disperse Blue 3

Disperse Red 11

Acid Blue 62

Disperse Blue 7

Basic Blue 22

Disperse Violet 15

Basic Blue 99

and also the following compounds:
1-N-methylmorpholiniumpropylamino-4-hydroxyanthraquinone
1-aminopropylamino-4-methylaminoanthra-quinone
1-aminopropylaminoanthraquinone
5-β-hydroxyethyl-1,4-diaminoanthraquinone
2-aminoethylaminoanthraquinone
1,4-bis(β,γ-dihydroxypropylamino)anthraquinone.

Among the azine dyes that may be mentioned are the following compounds:

Basic Blue 17

Basic Red 2.

Among the triarylmethane dyes that may be used according to the invention, mention may be made of the following compounds:

Basic Green 1

Acid Blue 9

Basic Violet 3

Basic Violet 14

Basic Blue 7

Acid Violet 49

Basic Blue 26

Acid Blue 7.

Among the indoamine dyes that may be used according to the invention, mention may be made of the following compounds:
2-β-hydroxyethylamino-5-[bis(β-4′-hydroxyethyl)amino]anilino-1,4-benzoquinone;
2-β-hydroxyethylamino-5-(2′-methoxy-4′-amino)anilino-1,4-benzoquinone;
3-N-(2′-chloro-4′-hydroxy)phenylacetyl-amino-6-methoxy-1,4-benzoquinoneimine;
3-N-(3′-chloro-4′-methylamino)phenylureido-6-methyl-1,4-benzoquinoneimine;
3-[4′-N-(ethylcarbamylmethyl)amino]phenylureido-6-methyl-1,4-benzoquinoneimine.

Among the dyes of tetraazapentamethine type that may be used according to the invention, mention may be made of the following compounds given in the table below, An being defined as above:

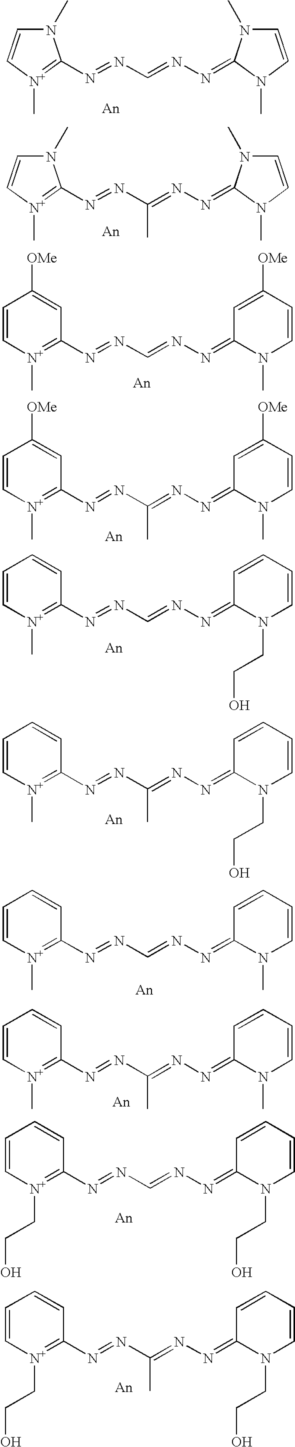

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin and apigenidin. Extracts or decoctions containing these natural dyes may also be used, and especially henna-based poultices or extracts.

When they are present, the amount of additional direct dye(s) in the composition generally ranges from 0.001% to 20% by weight relative to the weight of the composition and preferably from 0.01% to 10% by weight relative to the weight of the composition.

The medium that is suitable for dyeing, also known as the dye vehicle, generally consists of water or of a mixture of water and of at least one organic solvent to dissolve the compounds that would not be sufficiently water-soluble.

More particularly, the organic solvents are selected from linear or branched, preferably saturated monoalcohols or diols containing 2 to 10 carbon atoms, such as ethyl alcohol, isopropyl alcohol, hexylene glycol (2-methyl-2,4-pentanediol), neopentyl glycol and 3-methyl-1,5-pentanediol; aromatic alcohols such as benzyl alcohol and phenylethyl alcohol; glycols or glycol ethers, for instance ethylene glycol monomethyl, monoethyl and monobutyl ether, propylene glycol and its ethers, for instance propylene glycol monomethyl ether, butylene glycol and dipropylene glycol; and also diethylene glycol alkyl ethers, especially the C₁-C₄ ethers, for instance diethylene glycol monoethyl ether or monobutyl ether, alone or as a mixture.

The usual solvents described above, when they are present, usually represent from 1% to 40% by weight and more preferably from 5% to 30% by weight, relative to the total weight of the composition.

The dyeing composition in accordance with the invention may also include various adjuvants conventionally used in compositions for dyeing the hair, such as anionic, cationic, nonionic, amphoteric or zwitterionic surfactants or mixtures thereof, anionic, cationic, nonionic, amphoteric or zwitterionic polymers or mixtures thereof, mineral or organic thickeners, and in particular anionic, cationic, nonionic and amphoteric polymeric associative thickeners, antioxidants, penetrants, sequestrants, fragrances, buffers, dispersants, conditioning agents, for instance silicones, which may or may not be volatile or be modified, film-forming agents, ceramides, preservatives and opacifiers.

These adjuvants above are generally present in an amount for each of them of between 0.01% and 20% by weight relative to the weight of the composition.

The person skilled in the art will of course take care to select this or these optional additional compounds such that the advantageous properties intrinsically associated with the oxidation dyeing composition in accordance with the invention are not, or not substantially, adversely affected by the envisaged addition(s).

The pH of the dyeing composition in accordance with the invention is generally between about 3 and 12 and preferably between about 5 and 11. It may be adjusted to the desired value using acidifying or alkalifying agents usually used in the dyeing of keratin fibres, or alternatively using standard buffer systems.

Among the acidifying agents that may be mentioned, for example, are mineral or organic acids such as hydrochloric acid, orthophosphoric acid, sulphuric acid, carboxylic acids such as acetic acid, tartaric acid, citric acid and lactic acid, and sulphonic acids.

Among the alkalifying agents that may be mentioned, for example, are aqueous ammonia, alkaline carbonates, alkanolamines such as monoethanolamine, diethanolamine and triethanolamine and derivatives thereof, sodium hydroxide, potassium hydroxide and the compounds having the following formula: 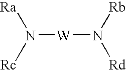
in which W is a propylene residue optionally substituted by a hydroxyl group or a C₁-C₄ alkyl radical; Ra, Rb, Rc and Rd, which are identical or different, represent a hydrogen atom or a C₁-C₄ alkyl or C₁-C₄ hydroxyalkyl radical.

The dyeing composition according to the invention may be in various forms, such as in the form of liquids, creams or gels, or in any other form that is suitable for dyeing keratin fibres, and especially human hair.

The composition according to the invention may further comprise at least one oxidizing agent. In this case, the composition is referred to as a ready-to-use composition.

For the purposes of the present invention, a ready-to-use composition is a composition intended to be applied immediately to the keratin fibres, i.e. it may be stored in unmodified form before use or may result from the extemporaneous mixing of two or more compositions.

Said composition may also be obtained by mixing the composition according to the invention with an oxidizing composition.

The oxidizing agent may be any oxidizing agent conventionally used in the field. Thus it may be selected from hydrogen peroxide, urea peroxide, alkali metal bromates, persalts such as perborates and persulphates, and also enzymes, among which mention may be made of peroxidases, 2-electron oxidoreductases such as uricases, and 4-electron oxygenases, for instance laccases. The use of hydrogen peroxide is particularly preferred.

The amount of oxidizing agent is generally between 1% and 40% by weight, relative to the weight of the ready-to-use composition, and preferably between 1% and 20% by weight relative to the weight of the ready-to-use composition.

Generally, the oxidizing composition used is an aqueous composition and may be in the form of a solution or an emulsion.

Usually, the composition free of oxidizing agent is mixed with about 0.5 to 10 weight equivalents of the oxidizing composition.

It should be noted that the pH of the ready-to-use composition is more particularly between 4 and 12 and preferably between 7 and 11.5.

The pH of the composition may be adjusted using an acidifying or alkalifying agent selected especially from those mentioned previously in the context of the description according to the invention.

The invention further provides a method of colouring that comprises the application of a dyeing composition according to the invention to the wet or dry keratin fibres.

The application to the fibres of the dyeing composition comprising the compound(s) of formula (I) or the addition salts thereof with an acid, optionally at least one oxidation base optionally combined with at least one coupler, and optionally at least one additional direct dye, may be performed in the presence of an oxidizing agent.

This oxidizing agent may be added to the composition comprising the compound(s) of formula (I) and the optional oxidation bases, couplers and/or additional direct dyes, either at the time of use or directly on the keratin fibre.

The oxidizing composition may also include various adjuvants conventionally used in compositions for dyeing the hair and as defined above.

The pH of the oxidizing composition containing the oxidizing agent is such that, after mixing with the dye composition, the pH of the resulting composition applied to the keratin fibres preferably ranges between 4 and 12 approximately and even more preferably between 7 and 11.5. It may be adjusted to the desired value by means of acidifying or alkalifying agents usually used in the dyeing of keratin fibres and as defined above.

The composition that is finally applied to the keratin fibres may be in various forms, such as in the form of liquids, creams or gels or in any other form that is suitable for dyeing keratin fibres, and especially human hair.

According to one particular embodiment, the composition according to the invention is free of oxidation base and of coupler.

The composition applied may optionally comprise at least one oxidizing agent.

The composition is thus applied to the wet or dry keratin fibres and is then left for a leave-in time that is sufficient to give the desired coloration.

Whatever the version adopted (with or without oxidizing agent), the leave-in time is generally between a few seconds and one hour, preferably between 3 and 30 minutes.

The temperature at which the composition is left to act is generally between 15 and 220° C., more particularly between 15 and 80° C. and preferably between 15 and 40° C.

After the leave-in time, the composition is preferably removed by rinsing with water, optionally followed by washing with a shampoo, and then optionally by drying.

Another subject of the invention is a device having a plurality of compartments or dyeing kit, in which a first compartment contains the dyeing composition of the invention and a second compartment contains the oxidizing composition. This device may be equipped with a means for delivering the desired mixture to the hair, such as the devices described in patent FR-2 586 913.

The examples that follow serve to illustrate the invention without, however, being limiting in nature.

### EXAMPLES

1—Synthesis of Compound 3: 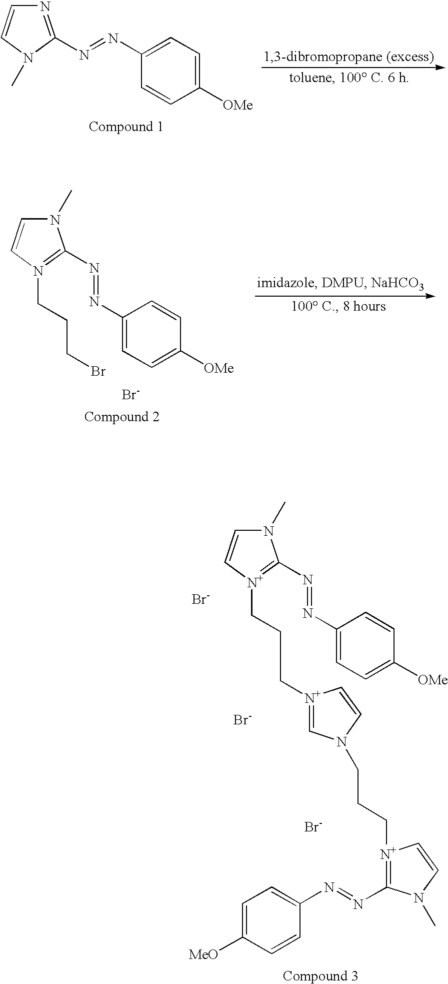
Compound 1 is Obtained in 3 Steps:
0—reaction of the diazonium salt of p-hydroxyaniline in the presence of imidazole.
1—O-alkylation reaction in the presence of methanol and hot sulphuric acid.
2—N-alkylation reaction in the presence of dimethyl carbonate and K₂CO₃.
   Step 1

Compound 1 (2.16 g) is reacted in the presence of 5.1 ml of 1,3-dibromopropane in 20 ml of toluene at 100° C. for 6 hours. The reaction mixture, which has been brought to ambient temperature beforehand, is poured into 100 ml of ethyl acetate. An orange-coloured precipitate is formed. After the resulting precipitate has been washed a number of times with ethyl acetate, it is isolated by filtration and then dried under vacuum. This gives an orange-coloured powder corresponding to the expected compound 2.

The ¹H NMR and mass analyses are in accordance with the expected product.

Step 2

Compound 2 (0.20 g) is reacted in the presence of 20 mg of imidazole and 25 mg of sodium hydrogencarbonate in 3 ml of DMPU at 100° C. for 6 hours. The reaction mixture, which is brought to ambient temperature beforehand, is poured into 50 ml of ethyl acetate. An orange-coloured precipitate is formed. The resulting precipitate is subsequently purified by liquid chromatography to give compound 3, which is obtained in the form of an orange-coloured powder.

The ¹H NMR and mass analyses are in accordance with the expected product.

2—Colouring Under Non-Oxidizing Conditions

5×10⁻⁴ mol of compound 2, obtained above, are dissolved in 5 ml of a mixture of water (2.5 ml) and pH 10 buffer (2.5 ml) with the following composition: 2 g of ammonium acetate

40 ml of water

NH₃ at 20% to pH 9-10

water to 100 ml

100 g of the above composition are applied to the hair at ambient temperature for 30 minutes. The hair is subsequently rinsed with water and dried.

The hair is coloured orange.

What is claimed is:

1. Symmetrical cationic diazo compounds of formula (I) below, their resonance forms, and their acid addition salts and/or solvates: 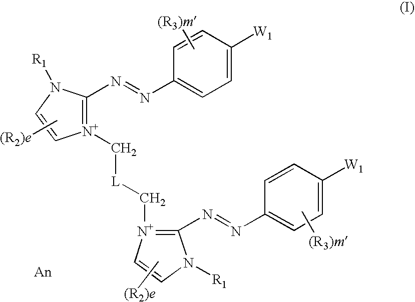
in which formula:
the radicals R₁, which are identical or not, represent:
an optionally substituted C₁-C₄ alkyl radical;
an optionally substituted phenyl radical;
an optionally substituted benzyl radical;
the radicals R₂, which are identical or not, represent:
an optionally substituted C₁-C₁₆ alkyl radical optionally interrupted by one or more heteroatoms and/or by one or more groups containing at least one heteroatom and selected preferably from oxygen, nitrogen, sulphur, —CO—, —SO₂— or combinations thereof, said alkyl radical being further optionally substituted by one or more groups selected from thio (—SH), C₁-C₄ thioalkyl; C₁-C₄ alkylsulphinyl or C₁-C₄ alkylsulphonyl groups;
a hydroxyl group,
a C₁-C₄ alkoxy group,
a C₂-C₄ (poly)hydroxyalkoxy group;
an alkoxycarbonyl group (RO—CO—) in which R represents a C₁-C₄ alkyl radical,
an alkylcarbonyloxy radical (RCO—O—) in which R represents a C₁-C₄ alkyl radical,
an alkylcarbonyl radical (R—CO—) in which R represents a C₁-C₄ alkyl radical,
an amino group,
an amino group substituted by one or two identical or different C₁-C₄ alkyl radicals optionally carrying at least one hydroxyl group, it being possible for the two alkyl radicals optionally to form, with the nitrogen atom to which they are attached, a heterocycle containing 1 to 3 heteroatoms, preferably 1 to 2 heteroatoms, selected from N, O and S, preferably N, and containing 5 to 7 ring members, which is saturated or unsaturated, aromatic or non-aromatic and is optionally substituted;
an alkylcarbonylamino group (RCO—NR′—) in which the radical R represents a C₁-C₄ alkyl radical and the radical R′ represents hydrogen or a C₁-C₄ alkyl radical;
an aminocarbonyl group ((R)₂N—CO—) in which the radicals R independently of one another represent a hydrogen atom or a C₁-C₄ alkyl radical;
a ureido group (N(R)₂—CO—NR′—) in which the radicals R and R′ independently of one another represent a hydrogen atom or a C₁-C₄ alkyl radical;
an aminosulphonyl group ((R)₂N—SO₂—) in which the radicals R independently of one another represent a hydrogen atom or a C₁-C₄ alkyl radical;
an alkylsulphonylamino group (RSO₂—NR′—) in which R represents a C₁-C₄ alkyl radical and R′ represents a hydrogen atom or a C₁-C₄ alkyl radical;
an optionally substituted aryl radical;
an optionally substituted (C₁-C₄)alkylaryl radical;
an alkylsulphinyl group (R—SO—) in which R represents a C₁-C₄ alkyl radical;
an alkylsulphonyl group (R—SO₂—) in which R represents a C₁-C₄ alkyl radical;
a nitro group;
a cyano group;
a halogen atom, preferably chlorine or fluorine;
a thio group (HS—);
an alkylthio group (RS—) in which the radical R represents an optionally substituted C₁-C₄ alkyl radical;
when e is 2, the two radicals R₂ may optionally form, with the carbon atoms to which they are attached, a secondary ring, aromatic or non-aromatic, containing 5 or 6 ring members, preferably 6 ring members, which is optionally substituted by one or more identical or non-identical groups selected from hydroxyl, C₁-C₄ alkyl, C₁-C₄ alkoxy, C₂-C₄ (poly)hydroxyalkoxy, amino, amino substituted by one or two identical or different C₁-C₄ alkyl radicals which optionally carry at least one hydroxyl group;
e is an integer from 0 to 2; when e is less than 2, the unsubstituted carbon atom(s) of the heterocycle carry a hydrogen atom,
the radicals R₃, which are identical or not, represent:
an optionally substituted C₁-C₁₆ alkyl radical optionally interrupted by one or more heteroatoms or by one or more groups containing at least one heteroatom and selected preferably from oxygen, nitrogen, sulphur, —CO—, —SO₂— or combinations thereof,
a hydroxyl group,
a C₁-C₄ alkoxy group,
a C₂-C₄ (poly)hydroxyalkoxy group;
an alkoxycarbonyl group (RO—CO—) in which R represents a C₁-C₄ alkyl radical,
an alkylcarbonyloxy radical (RCO—O—) in which R represents a C₁-C₄ alkyl radical;
an alkylcarbonyl radical (R—CO—) in which R represents a C₁-C₄ alkyl radical;
an amino group;
an amino group substituted by one or two identical or different C₁-C₄ alkyl radicals optionally carrying at least one hydroxyl group; it being possible for the two alkyl radicals optionally to form, with the nitrogen atom to which they are attached, a heterocycle containing 1 to 3 heteroatoms, preferably 1 to 2 heteroatoms, selected from N, O and S, preferably N, and containing 5 to 7 ring members, which is saturated or unsaturated, aromatic or non-aromatic and is optionally substituted;
an alkylcarbonylamino group (RCO—NR′—) in which the radical R represents a C₁-C₄ alkyl radical and the radical R′ represents a hydrogen atom or a C₁-C₄ alkyl radical;
an aminocarbonyl group ((R)₂N—CO—) in which the radicals R independently of one another represent a hydrogen atom or a C₁-C₄ alkyl radical;
a ureido group (N(R)₂—CO—NR′—) in which the radicals R and R′ independently of one another represent a hydrogen atom or a C₁-C₄ alkyl radical;
an aminosulphonyl group ((R)₂N—SO₂—) in which the radicals R independently of one another represent a hydrogen atom or a C₁-C₄ alkyl radical;
an alkylsulphonylamino group (RSO₂—NR′—) in which the radicals R and R′ independently of one another represent a hydrogen atom or a C₁-C₄ alkyl radical;
a thio group (HS—);
an alkylthio group (RS—) in which the radical R represents a C₁-C₄ alkyl radical;
an alkylsulphinyl group (R—SO—) in which R represents a C₁-C₄ alkyl radical;
an alkylsulphonyl group (R—SO₂—) in which R represents a C₁-C₄ alkyl radical;
a nitro group;
a cyano group;
a halogen atom, preferably chlorine or fluorine;
when m′ is greater than or equal to 2, two adjacent radicals R₃ may form, with the carbon atoms to which they are attached, a secondary ring, aromatic or non-aromatic, containing 6 ring members, which is optionally substituted by one or more groups selected from the following groups: hydroxyl, C₁-C₄ alkyl, C₁-C₄ alkoxy, C₂-C₄ (poly)hydroxyalkoxy, C₁-C₄ alkylcarbonylamino, amino, amino substituted by one or two identical or different C₁-C₄ alkyl radicals which optionally carry at least one hydroxyl group,
m′ is an integer from 0 to 4; when m′ is less than 4, then the unsubstituted carbon atom(s) of the aromatic ring carry a hydrogen atom;
W₁ radicals, which are identical, represent:
a hydrogen atom,
a halogen atom selected from bromine, chlorine and fluorine, preferably chlorine and fluorine,
an —NR₅R₆, OR₇, —NR₄—Ph—NR₅R₆, —NR₄—Ph—OR₇, —O—Ph—OR₇ or —O—Ph—NR₅R₆ group, where:
R₄ and R₇, which are identical or not, represent a hydrogen atom, an optionally substituted C₁-C₂₀, preferably C₁-C₁₆, alkyl radical, an optionally substituted C₁-C₃ aralkyl radical or an optionally substituted phenyl radical;
R₅ and R₆, which are identical or not, represent a hydrogen atom, an optionally substituted C₁-C₂₀, preferably C₁-C₁₆, alkyl radical, an optionally substituted phenyl radical, an optionally substituted C₁-C₃ aralkyl radical or an alkylcarbonyl radical (R—CO—) in which R is a C₁-C₄ alkyl radical;
R₅ and R₆ may optionally form, with the nitrogen atom to which they are attached, a heterocycle containing 1 to 3 heteroatoms, preferably 1 or 2 heteroatoms, selected from N, O and S, preferably N, and containing 5 to 7 ring members, which is saturated or unsaturated, aromatic or non-aromatic and is optionally substituted;
R₅ and R₆, independently of one another, may form, with the carbon atom of the aromatic ring adjacent to that to which —NR₅R₆ is attached, a 5- or 6-membered saturated heterocycle;
Ph represents an optionally substituted phenyl radical;
L, a cationic linker connecting the two identical azo chromophores, represents a C₂-C₄₀ alkyl radical which carries at least one cationic charge and is optionally substituted and/or optionally interrupted by one or more saturated or unsaturated, aromatic or non-aromatic, identical or different (hetero)cycles containing 3 to 7 ring members and/or optionally interrupted by one or more heteroatoms or groups containing at least one heteroatom, or combinations thereof, such as, for example, oxygen, nitrogen, sulphur, —CO—, —SO₂— or combinations thereof, the linker L containing no azo, nitro, nitroso or peroxo bond; with the proviso that the linker L carries at least one cationic charge;
the electroneutrality of the compound of formula (I) being ensured by one or more identical or non-identical, cosmetically acceptable anions An.

2. Compounds according to one of the preceding claims, characterized in that the radicals R₁ represent:
a C₁-C₄ alkyl radical which is optionally substituted by one or more radicals selected from hydroxyl, C₁-C₂ alkoxy, amino and C₁-C₂ (di)alkylamino radicals;
an optionally substituted benzyl radical.

3. Compounds according to either of the preceding claims, characterized in that the radicals R₂, which are identical or not, represent:
a halogen atom selected from chlorine and fluorine;
a C₁-C₄ alkyl radical optionally substituted by one or more identical or different radicals selected from hydroxyl, C₁-C₂ alkoxy, C₂-C₄ (poly)hydroxyalkoxy, amino, C₁-C₂ (di)alkylamino, thio (—SH), C₁-C₄ alkylsulphinyl, C₁-C₄ alkylsulphonyl and C₁-C₄ thioalkyl radicals;
a phenyl radical optionally substituted by one or more identical or different radicals selected from hydroxyl, C₁-C₂ alkoxy, C₂-C₄ (poly)hydroxyalkoxy, amino and C₁-C₂ (di)alkylamino radicals or a halogen atom such as chlorine or fluorine;
a C₁-C₄ alkoxy radical;
a C₁-C₄ alkylsulphonylamino radical;
a C₂-C₄ (poly)hydroxyalkoxy radical;
an amino radical;
a C₁-C₂ (di)alkylamino radical;
a C₂-C₄ (poly)hydroxyalkylamino radical;
an alkylsulphonylamino radical (RSO₂N—) in which the radical R represents a C₁-C₄ alkyl radical;
an aminosulphonyl radical ((R)₂NSO₂—) in which the radicals R independently of one another represent a hydrogen atom or a C₁-C₄ alkyl radical;
an alkylthio radical (RS—) in which the radical R represents a C₁-C₄ alkyl radical;
an alkylsulphinyl radical (RSO—) in which the radical R represents a C₁-C₄ alkyl radical;
an alkylsulphonyl radical (R—SO₂—) in which the radical R represents a C₁-C₄ alkyl radical;
an alkylcarbonylamino radical (RCONR′—) in which the radical R represents a hydrogen atom or a C₁-C₄ alkyl radical and the radical R′ represents a hydrogen atom or a C₁-C₄ alkyl radical.

4. Compounds according to one of the preceding claims, characterized in that the two radicals R₂ may optionally form, with the carbon atoms to which they are attached, a secondary, 6-membered aromatic ring optionally substituted by one or more identical or different groups selected from hydroxyl, C₁-C₄ alkyl, C₁-C₄ alkoxy, amino, and (di)(C₁-C₄)-alkylamino which optionally carry at least one hydroxyl or methylcarbonylamino group.

5. Compounds according to one of the preceding claims, characterized in that the radicals R₃, which are identical or different, represent:
an optionally substituted C₁-C₁₆, preferably C₁-C₈ alkyl radical;
a halogen atom such as chlorine or fluorine;
a hydroxyl group;
a C₁-C₂ alkoxy radical;
a C₂-C₄ (poly)hydroxyalkoxy radical;
an amino radical;
an amino radical substituted by one or two identical or different C₁-C₄ alkyl radicals which optionally carry at least one hydroxyl group or at least one C₁-C₄ alkoxy radical, it being possible for the two alkyl radicals to form, with the nitrogen atom to which they are attached, a heterocycle containing 1 to 3 heteroatoms, preferably 1 or 2 heteroatoms, selected from N, O and S, preferably N, the heterocycle containing 5 to 7 ring members, being saturated or unsaturated, aromatic or non-aromatic, and being optionally substituted;
an alkylcarbonylamino radical (RCO—NR′—) in which the radical R represents a C₁-C₄ alkyl radical and the radical R′ represents a hydrogen or a C₁-C₄ alkyl radical;
an alkylsulphonylamino radical (R′SO₂—NR—) in which the radical R represents a hydrogen atom or a C₁-C₄ alkyl radical and the radical R′ represents a C₁-C₄ alkyl radical;
an aminosulphonyl radical ((R)₂N—SO₂—) in which the radicals R, which are identical or not, represent a hydrogen atom or a C₁-C₄ alkyl radical;
an alkylthio radical (RS—) in which the radical R represents a C₁-C₄ alkyl radical;
an alkylsulphonyl radical (R—SO₂—) in which the radical R represents a C₁-C₄ alkyl radical.

6. Compounds according to one of the preceding claims, characterized in that the radicals R₃, which are identical or different, represent:
a C₁-C₄ alkyl radical which is optionally substituted by one or more radicals selected from hydroxyl radicals, C₁-C₂ alkylcarbonylamino radicals, amino radicals substituted by two identical or different C₁-C₂ alkyl radicals which optionally carry at least one hydroxyl group or a C₁-C₂ alkoxy radical, it being possible for these two alkyl radicals optionally to form, with the nitrogen atom to which they are attached, a 5- or 6-membered heterocycle which is saturated or unsaturated and is optionally aromatic, selected preferably from pyrrolidine, piperazine, homopiperazine, pyrrole, imidazole and pyrazole;
a C₂-C₄ hydroxyalkoxy radical;
a halogen selected from chlorine and fluorine;
an amino radical;
an amino radical substituted by one or two identical or different C₁-C₂ alkyl radicals which optionally carry at least one hydroxyl group;
a methylcarbonylamino radical;
a methylsulphonylamino radical;
a hydroxyl radical;
a C₁-C₂ alkoxy radical;
a methylsulphonyl radical.

7. Compounds according to one of the preceding claims, characterized in that, when the coefficient m′ is greater than or equal to 2, two adjacent radicals R₃ may form, with the carbon atoms to which they are attached, a secondary, 6-membered aromatic ring optionally substituted by one or more groups selected from hydroxyl groups, —NR₄—Ph, —NR₄—Ph—NR₅R₆ and —NR₄—Ph—OR₇ groups, C₁-C₄ alkyl groups, C₁-C₄ alkoxy groups, C₂-C₄ (poly)hydroxyalkoxy groups, C₁-C₄ alkylcarbonylamino groups, amino groups, and (di)alkylamino groups in which the C₁-C₄ alkyl radical(s) optionally carry at least one hydroxyl group.

8. Compounds according to the preceding claim, characterized in that two adjacent radicals R₃ may form, with the carbon atoms to which they are attached, a secondary, 6-membered aromatic ring which is optionally substituted by one or more groups selected from hydroxyl, methoxy, ethoxy, 2-hydroxyethyloxy, amino, methylcarbonylamino, (di)-2-hydroxyethylamino, —NH—Ph, —NH—Ph—NH₂, —NH—Ph—NHCOCH₃, —NH—Ph—OH and —NH—Ph—OCH₃ groups.

9. Compounds according to the preceding claim, characterized in that R₄ and R₇ independently of one another represent:
a hydrogen atom;
a C₁-C₆ alkyl radical which is optionally substituted by one or more identical or different groups selected from hydroxyl and C₁-C₂ alkoxy;
an aryl or arylalkyl radical, such as phenyl or benzyl, the aryl moiety being optionally substituted by one or more identical or different groups selected from chlorine, amino, hydroxyl, C₁-C₂ alkoxy, amino which is mono- or disubstituted by two identical or different radicals selected from C₁-C₄ alkyl radicals which optionally carry at least one hydroxyl group.

10. Compounds according to one of the preceding claims, characterized in that the radicals R₅ and R₆, which are identical or different, represent:
a hydrogen atom;
an alkylcarbonyl radical (R—CO—) in which R represents an optionally substituted C₁-C₄ alkyl radical;
a C₁-C₆ alkyl radical which is optionally substituted, preferably by at least one hydroxyl group, at least one C₁-C₂ alkoxy group, at least one amino group, at least one C₁-C₄ (di)alkylamino group; the alkyl radical may further be substituted by one or more identical or different groups selected from C₁-C₄ alkylsulphonyl, C₁-C₄ alkylsulphinyl and C₁-C₄ alkylcarbonyl;
an aryl or arylalkyl radical, such as phenyl or benzyl, the aryl moiety being optionally substituted by one or more identical or different groups selected from chlorine, amino, hydroxyl, C₁-C₄ alkoxy, amino which is mono- or disubstituted by two identical or different radicals selected from C₁-C₄ alkyl radicals which optionally carry at least one hydroxyl group.

11. Compounds according to one of the preceding claims, characterized in that the radicals R₅ and R₆, which are identical or different, represent:
a hydrogen atom;
a methylcarbonyl, ethylcarbonyl or propylcarbonyl radical;
an optionally substituted C₁-C₃ alkyl radical, such as methyl, ethyl, 2-hydroxyethyl or 2-methoxyethyl;
a phenyl radical which is optionally substituted by one or more radicals selected from hydroxyl radicals, C₁-C₂ alkoxy radicals, amino radicals, amino radicals substituted by one or more C₁-C₄ groups which optionally carry at least one hydroxyl group.

12. Compounds according to one of claims 1 to 11, characterized in that the radicals R₅ and R₆ form, together with the nitrogen atom to which each is attached, a heterocycle containing 1 to 3 heteroatoms, preferably 1 or 2 heteroatoms, selected from N, O and S, preferably N, and containing 5 to 7 ring members, which is saturated or unsaturated, aromatic or non-aromatic, and is optionally substituted.

13. Compounds according to the preceding claim, characterized in that the heterocycle containing 5 to 7 ring members is selected from the following heterocycles: piperidine, 2-(2-hydroxyethylpiperidine), 4-(aminomethyl)piperidine, 4-(2-hydroxyethyl)piperidine, 4-(dimethylamino)piperidine, piperazine, 1-methylpiperazine, 1-(2-hydroxyethyl)piperazine, 1-(2-aminoethyl)piperazine, 1-hydroxyethylethoxypiperazine, homopiperazine, 1-methyl-1,4-perhydrodiazepine, pyrrole, 1,4-dimethylpyrrole, 1-methyl-4-ethylpyrrole, 1-methyl-4-propylpyrrole.

14. Compounds according to one of claims 1 to 11, characterized in that the radicals R₅ and R₆ represent alkyl radicals which, independently of one another, form, with the carbon atom of the aromatic ring optionally substituted by a hydroxyl and adjacent to that to which —NR₅R₆ is attached, a 5- or 6-membered saturated heterocycle.

15. Compounds according to one of the preceding claims, characterized in that L represents advantageously a C₂-C₂₀ alkyl radical:
1—interrupted by at least one group corresponding to the following formulae: 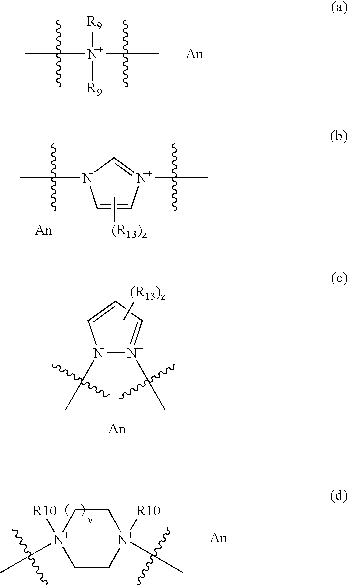
in which:
R₉ and R₁₀ independently of one another represent a C₁-C₈ alkyl radical; a C₁-C₆ monohydroxyalkyl radical; a C₂-C₆ polyhydroxyalkyl radical; a C₁-C₆ alkoxy-C₁-C₆ alkyl radical; an aryl radical such as phenyl which is optionally substituted; an arylalkyl radical such as benzyl which is optionally substituted; a C₁-C₆ aminoalkyl radical; a C₁-C₆ aminoalkyl radical whose amine is substituted by one or more identical or different C₁-C₄ alkyl radicals, or C₁-C₆ alkylsulphonyl;
two radicals R₉ may form, together with the nitrogen atom to which they are attached, an optionally substituted 5-, 6- or 7-membered saturated or unsaturated ring;
R₁₃ radicals, which are identical or different, represent a halogen atom selected from bromine, chlorine and fluorine, a C₁-C₆ alkyl radical, a C₁-C₆ monohydroxyalkyl radical, a C₂-C₆ polyhydroxyalkyl radical, a C₁-C₆ alkoxy radical, a C₁-C₄ (di)alkylamino radical, a hydroxycarbonyl radical, a C₁-C₆ alkylcarbonyl radical, a C₁-C₆ thioalkyl radical, a C₁-C₆ alkylthio radical, an amino radical (di)substituted by a C₁-C₆ alkyl radical, C₁-C₆ alkylsulphonyl, a benzyl radical which is optionally substituted, or a phenyl radical which is optionally substituted by one or more radicals selected from methyl, hydroxyl, amino and methoxy radicals;
An is an organic or inorganic anion or anion mixture;
z is an integer between 1 and 3; if z<3, the unsubstituted carbon atoms carry a hydrogen atom;
v is an integer 1 or 2, preferably 1;
2—optionally interrupted by one or more heteroatoms or groups containing at least one heteroatom, or combinations thereof, such as, for example, oxygen, nitrogen, sulphur, a —CO— group, a —SO₂— group; with the proviso that there is no nitro, nitroso or peroxo group or bond in the linker L;
3—and optionally substituted by one or more radicals selected from hydroxyl radicals, C₁-C₂ alkoxy radicals, C₂-C₄ (poly)hydroxyalkoxy radicals, and amino radicals substituted by one or more linear or branched C₁-C₂ alkyl groups which optionally carry at least one hydroxyl group.

16. Compounds according to any one of the preceding claims, characterized in that the anion An represents an organic or inorganic anion or anion mixture allowing the charge or charges on the compounds of formula (I) to be balanced, and selected for example from a halide such as chloride, bromide, fluoride or iodide; a hydroxide; a sulphate; a hydrogensulphate; an alkylsulphate for which the linear or branched alkyl moiety is C₁-C₆, such as the methylsulphate or ethylsulphate ion; carbonates and hydrogencarbonates; salts of carboxylic acids, such as formate, acetate, citrate, tartrate and oxalate; alkylsulphonates for which the linear or branched alkyl moiety is C₁-C₆, such as the methylsulphonate ion; arylsulphonates for which the aryl moiety, preferably phenyl, is optionally substituted by one or more C₁-C₄ alkyl radicals, such as 4-tolylsulphonate, for example; and alkylsulphonyls such as mesylate.

17. Compounds according to any one of the preceding claims, characterized in that they correspond to formula (I′) below, and also their resonance forms, their acid addition salts and/or their solvates: 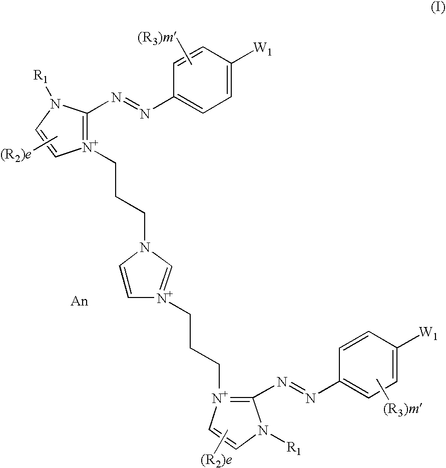
the groups W₁, the radicals R₁, R₂, R₃, the coefficients e and m′ being defined as before.

18. Compounds according to any one of the preceding claims, characterized in that they correspond to the following formulae, to their addition salts with an acid, or their solvates:

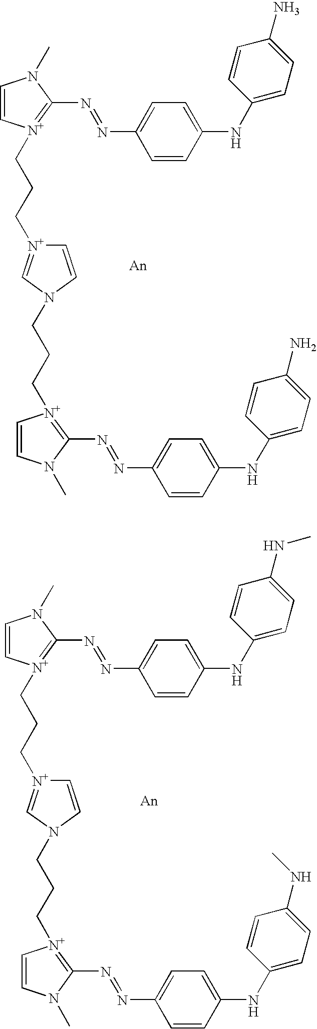

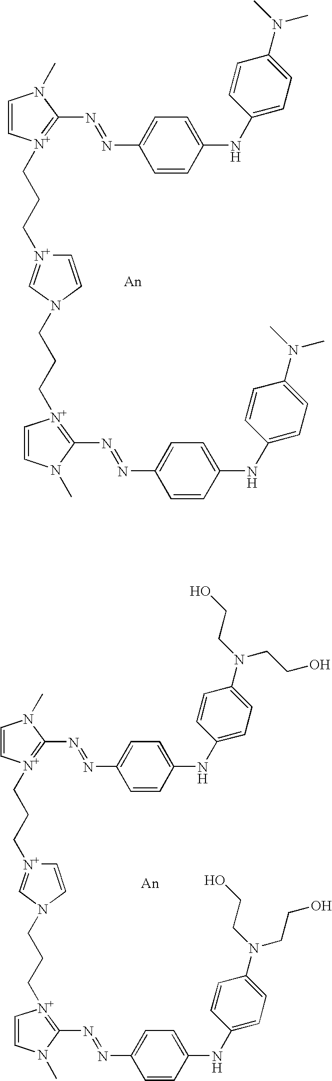

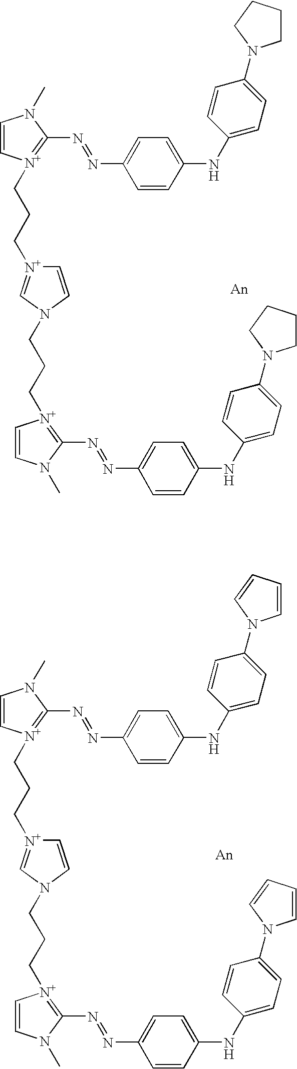

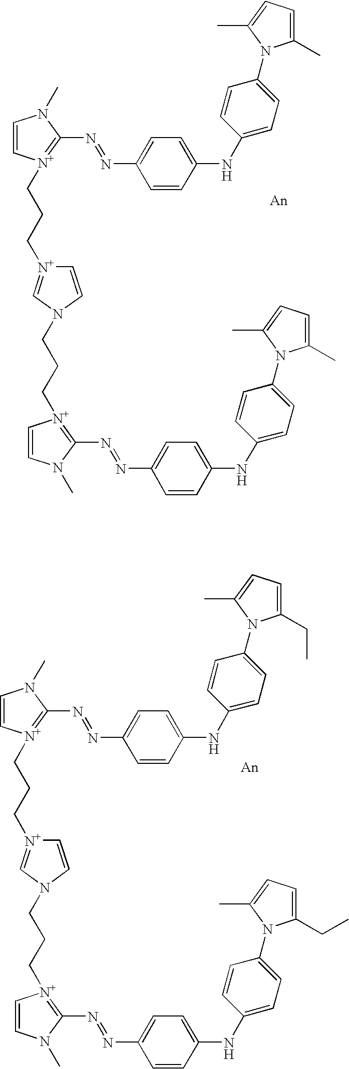

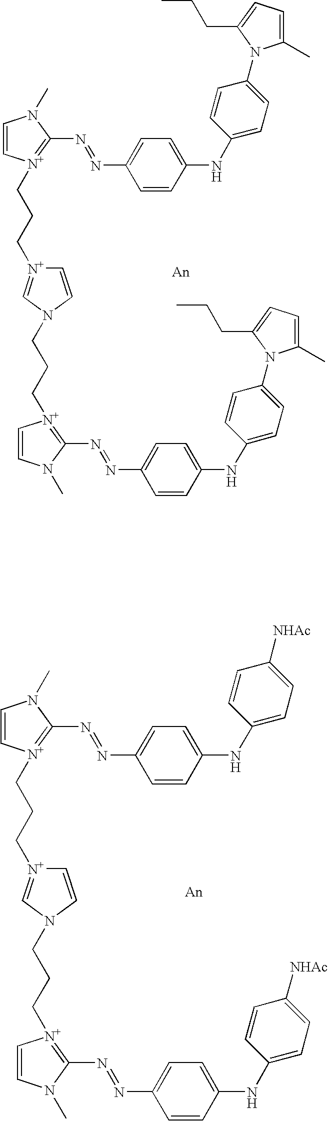

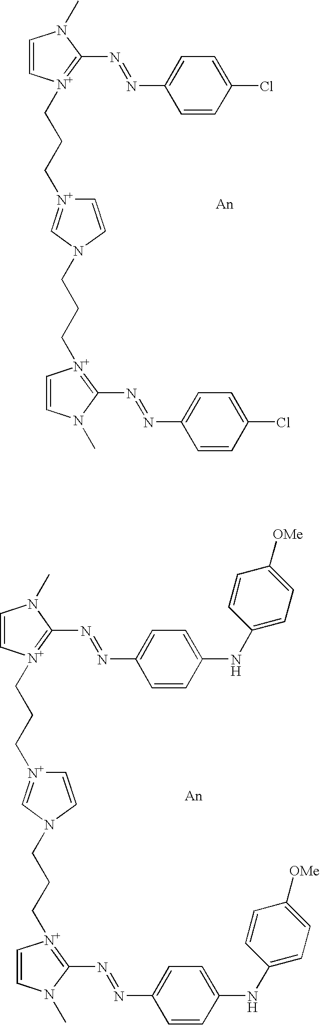

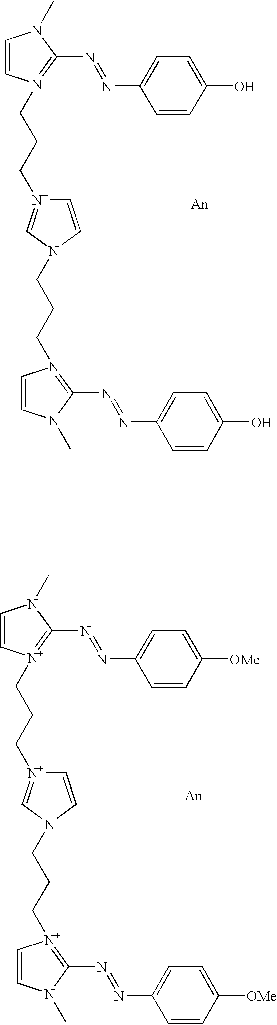

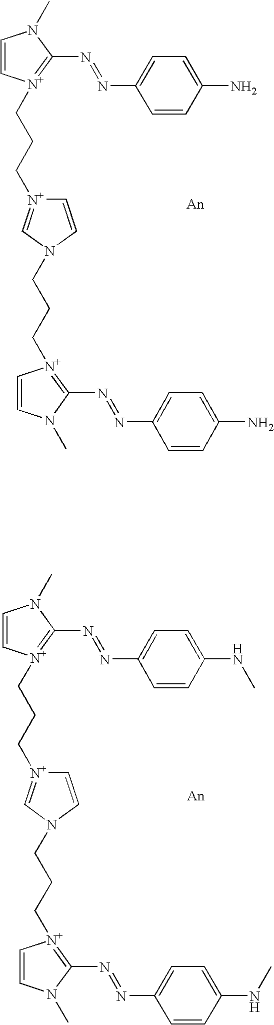

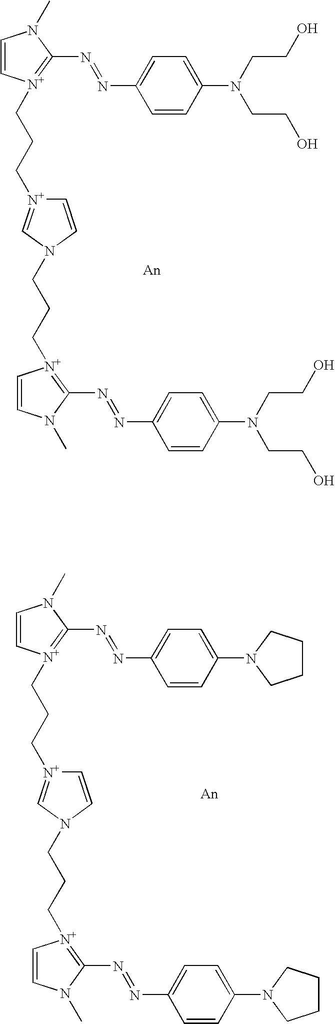

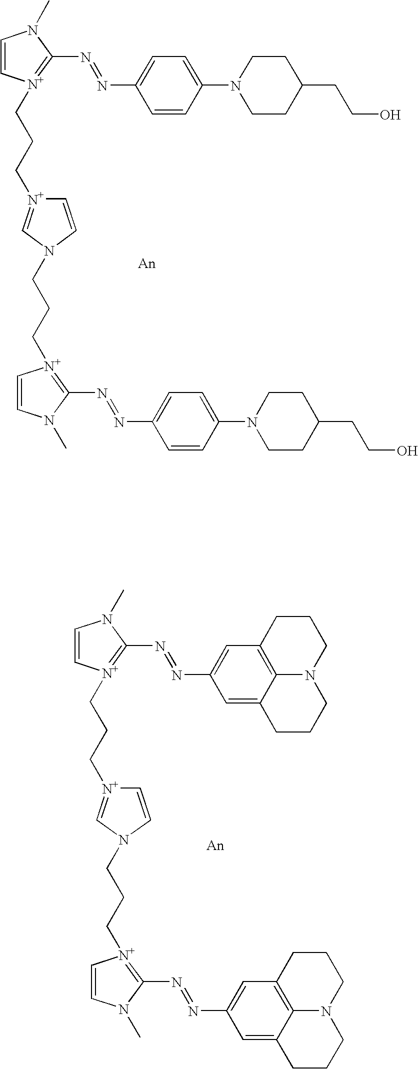

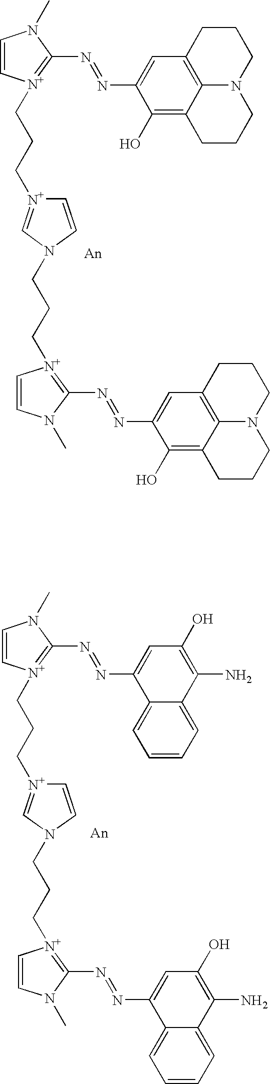

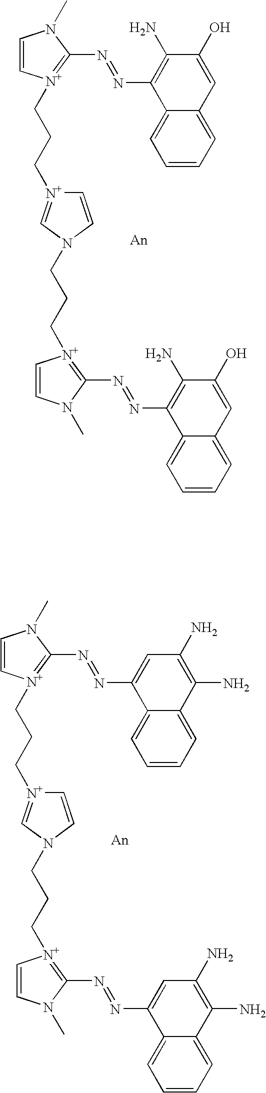

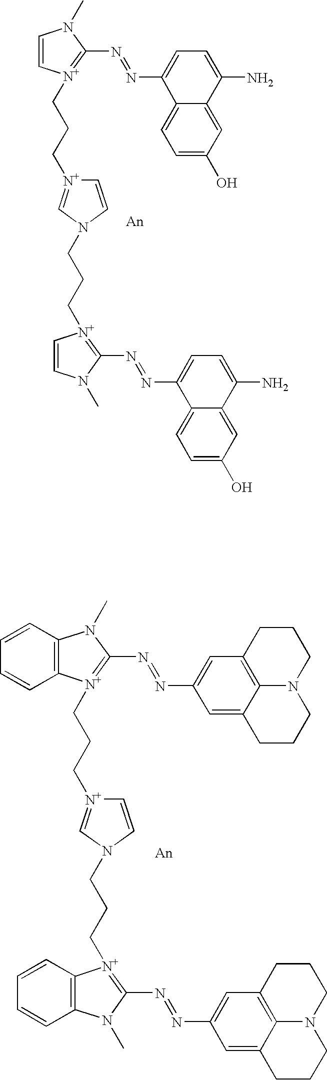

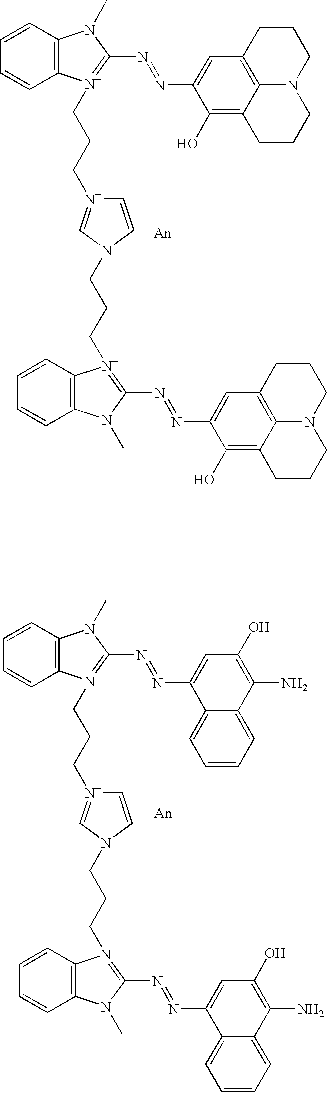

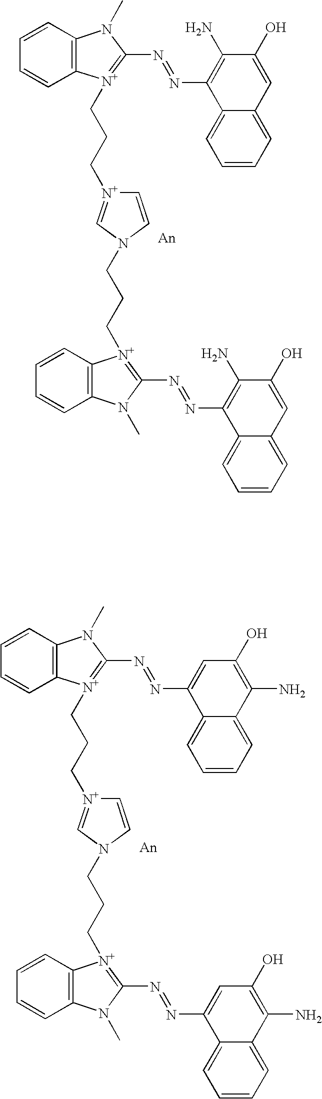

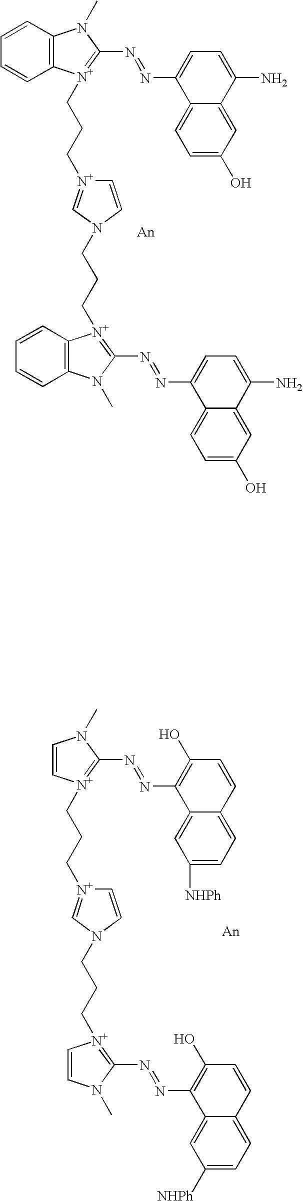

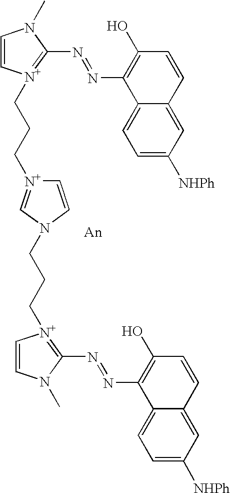

19. Dyeing composition comprising, in a medium appropriate for the dyeing of keratin fibres, as direct dye at least one compound of formula (I), or its addition salts with an acid, according to any one of the preceding claims.

20. Composition according to the preceding claim, characterized in that the amount of compound of formula (I) or of each of the compounds of formula (I) is between 0.001% and 20% by weight relative to the total weight of the dyeing composition, more particularly between 0.01% and 10% by weight.

21. Composition according to either of claims 19 and 20, characterized in that it comprises at least one additional direct dye, at least one oxidation base optionally in combination with at least one coupler, or mixtures thereof.

22. Composition according to the preceding claim, characterized in that the additional direct dye is a cationic or nonionic dye selected from nitrobenzene dyes, azo, azomethine, methine, tetraazapentamethine, anthraquinone, naphthoquinone, benzoquinone, phenothiazine, indigoid, xanthene, phenanthridine, phthalocyanine dyes, dyes derived from triarylmethane, and natural dyes, alone or as mixtures.

23. Composition according to claim 21, characterized in that the oxidation base is selected from o-phenylenediamines, p-phenylenediamines, double bases, o-aminophenols, p-aminophenols, heterocyclic bases, their acid addition salts and their mixtures.

24. Composition according to claim 21, characterized in that the coupler is selected from m-aminophenols, m-phenylenediamines, m-diphenols, naphthols, heterocyclic couplers, their acid addition salts and their mixtures.

25. Composition according to any one of claims 19 to 24, characterized in that it comprises at least one oxidizing agent.

26. Method of colouring keratin fibres which consists in contacting a composition according to any one of claims 19 to 24 with said fibres, which are dry or wet, for a time sufficient to give the desired effect.

27. Device having a plurality of compartments, in which a first compartment contains the composition according to any one of claims 19 to 23 and a second compartment contains an oxidizing composition.
